(19) United States
(12) Patent Application Publication
(10) Pub. No.: US 2026/0199084 A1
(43) Pub. Date: Jul. 16, 2026

(54) VALVES WITH REDUCED TISSUE INGROWTH AND PANNUS FORMATION

(71) Applicant: Edwards Lifesciences Corporation, Irvine, CA (US)

(72) Inventors: Biswa P. Das, Irvine, CA (US); Radhika Vaid, Rancho Mission Viejo, CA (US); Shrojalkumar M. Desai, Tustin, CA (US); Avina Gupta, Tustin, CA (US)

(21) Appl. No.: 19/563,120

(22) Filed: Mar. 11, 2026

Related U.S. Application Data
(63) Continuation of application No. PCT/US2024/046537, filed on Sep. 13, 2024.
(60) Provisional application No. 63/582,837, filed on Sep. 14, 2023.

(51) Int. Cl. A61F 2/24; A61L 27/18

(52) U.S. Cl. CPC A61F 2/2418; A61F 2/2415; A61L 27/18; A61F 2210/0076; A61F 2220/0075; A61L 2430/20

(57) ABSTRACT

Disclosed herein is an implantable prosthetic valve configured to substantially reduce pannus formation and/or uncontrolled tissue ingrowth along the implantable prosthetic valve. Also disclosed herein are methods of making such implantable prosthetic valves. Also disclosed herein are kits comprising one or more sutures configured to substantially reduce pannus formation and/or uncontrolled tissue ingrowth along the implantable prosthetic valve.

### CROSS-REFERENCE TO CORRELATED APPLICATION

This application is a continuation of International Application No. PCT/US2024/046537, filed September 13, 2024, which claims the benefit of U.S. Patent Application No. 63/582,837 filed September 14, 2023, the entire contents all of which are incorporated by reference for all purposes.

### FIELD

The present application concerns aspects of prosthetic valves with reduced tissue ingrowth and pannus formation.

### BACKGROUND

The human heart can suffer from various valvular diseases. These valvular diseases can result in significant malfunctioning of the heart and ultimately require the replacement of the native valve with an artificial valve. There are a number of known artificial valves and a number of known methods of implanting these artificial valves in humans.

When the native valve is replaced, surgical implantation of the prosthetic valve typically requires an open-chest surgery during which the heart is stopped, and the patient is placed on cardiopulmonary bypass (a so-called “heart-lung machine”). In one common surgical procedure, the diseased native valve leaflets are excised, and a prosthetic valve is sutured to the surrounding tissue at the valve annulus. Because of the trauma associated with the procedure and the attendant duration of extracorporeal blood circulation, some patients do not survive the surgical procedure or die shortly thereafter. It is well known that the risk to the patient increases with the amount of time required for extracorporeal circulation. Due to these risks, a substantial number of patients with defective native valves are deemed inoperable because their condition is too frail to withstand the procedure. By some estimates, more than 50% of the subjects suffering from valve stenosis who are older than 80 years cannot be operated on for valve replacement.

Because of the drawbacks associated with conventional open-heart surgery, percutaneous and minimally-invasive surgical approaches are garnering intense attention. In one technique, a prosthetic valve is configured to be implanted in a much less invasive procedure by way of catheterization. For instance, U.S. Pat. Nos. 5,411,522 and 6,730,118, which are incorporated herein by reference for all purposes, describe collapsible transcatheter heart valves that can be percutaneously introduced in a compressed state on a catheter and expanded in the desired position by balloon inflation or by utilization of a self-expanding frame or stent.

One of the challenges associated with implantable heart valves is the possibility of tissue growth and pannus growth on the inner surfaces of the valve, which can lead to device malfunction.

Therefore, there is a need for valves and materials that can substantially reduce tissue growth and ensure the high longevity of the device. These needs and other needs are at least partially satisfied by the present disclosure.

### SUMMARY

Aspects of the present disclosure relate to implantable prosthetic valves. Some aspects relate to an implantable prosthetic valve that comprises an annular frame having an inner surface and an outer surface, and wherein the frame has an inflow end and an outflow end, and a central longitudinal axis extending from the inflow end to the outflow end. In such aspects, the implantable prosthetic valve can further comprise an inner skirt having an inner surface and an outer surface and positioned along the inner surface of the frame. Still, further, the implantable prosthetic valve can comprise an optional a leaflet structure having an inner surface and an outer surface and attached to the inner surface of the annular frame. Still further, the implantable prosthetic valve can further comprise an optional outer skirt having an inner surface and an outer surface and positioned at the outer surface of the annular frame. The implantable prosthetic valve disclosed herein comprises one or more sutures, wherein the one or more sutures are configured to attach at least a portion of the inner skirt, at least a portion of the leaflet structure and/or at least a portion of the outer skirt, if present, to at least the annular frame and wherein the one or more sutures are configured to substantially reduce pannus formation and/or uncontrolled tissue ingrowth along the implantable prosthetic valve.

Still further disclosed herein is an implantable prosthetic valve comprising: an annular frame having an inner surface and an outer surface wherein the frame has an inflow end and an outflow end, and a central longitudinal axis extending from the inflow end to the outflow end; an inner skirt having an inner surface and an outer surface and positioned along the inner surface of the frame; wherein the inner surface of the inner skirt comprises a first pannus limiting material; an optional leaflet structure having an inner surface and an outer surface and attached to the inner surface of the annular frame; and/or an optional outer skirt having an inner surface and an outer surface and positioned at the outer surface of the annular frame, and one or more sutures, wherein the one or more sutures are configured to attach at least a portion of the inner skirt, at least a portion of the leaflet structure and/or at least a portion of the outer skirt, if present, to at least the annular frame and wherein the one or more sutures are configured to substantially reduce pannus formation and/or uncontrolled tissue ingrowth along the implantable prosthetic valve.

In some aspects, the one or more sutures have a substantially cylindrical form. In some aspects, the one or more sutures have a substantially flattened form.

In some aspects, at least a portion of a surface of the one or more sutures has a coating comprising a pannus limiting material. In such exemplary and unlimiting aspects, the pannus limiting material comprises thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

Also disclosed herein are aspects that encompass methods of making the disclosed implantable prosthetic valves. In one aspect, disclosed herein is the method of forming an implantable prosthetic valve comprising: attaching at least a portion of an inner skirt to at least an annular frame with one or more sutures such that an outer surface of the inner skirt is at least partially attached to an inner surface of the annular frame, wherein the one or more sutures are configured to substantially reduce pannus formation along the implantable prosthetic valve, and wherein an inner surface of the inner skirt faces an inner space formed by the annular frame; and wherein the annular frame has an inflow end and an outflow end, and a central longitudinal axis extending from the inflow end to the outflow end.

Also disclosed herein is a method for preventing tissue ingrowth, comprising inserting any of the disclosed herein implantable prosthetic valves in a patient’s body.

Also disclosed herein is a kit comprising one or more sutures configured to attach at least a portion of an inner skirt and/or at least a portion of a leaflet structure and/or at least a portion of an outer skirt to at least a frame of an implantable prosthetic valve, wherein the one or more sutures are configured to substantially reduce pannus formation and/or uncontrolled tissue ingrowth along the implantable prosthetic valve.

The foregoing and other features and advantages of the disclosure will become more apparent from the following detailed description, which proceeds with reference to the accompanying figures.

### BRIEF DESCRIPTION OF THE DRAWINGS

FIG. 1 shows exemplary locations for pannus growth in an exemplary prosthetic valve implanted in a patient’s body in one aspect.

FIGS. 2A-2B show a partial schematic of an exemplary implantable prosthetic valve with an exemplary placement of one or more sutures in one aspect.

FIG. 3 shows a diagram of an exemplary method of forming the one or more sutures in one aspect.

FIGS. 4A-4C show photographs of exemplary sutures. FIG. 4A shows a photograph of an untreated PET-based suture in one aspect. FIG. 4B shows a photograph of a PET-based suture configured to reduce pannus growth in one aspect. FIG. 4C shows a photograph of a PET-based suture configured to reduce pannus growth in other aspects.

FIGS. 5A-5B show photographs of exemplary sutures in one aspect. FIG. 5A shows a photograph of an untreated ultra-high molecular weight polyethylene- based suture in one aspect. FIG. 5B shows a photograph of an ultra-high molecular weight polyethylene-based suture configured to reduce pannus growth in one aspect.

FIGS. 6A-6E show SEM images of exemplary sutures in one aspect. FIG. 6A shows an exemplary untreated suture. FIG. 6B shoes the exemplary suture that was treated to reduce pannus growth. FIG. 6C shoes a close-up image of the suture in FIG. 6B. FIG. 6D shows the suture that was treated to further reduce the pannus growth in other aspects. FIG. 6E shows a cross section of the suture shown in FIG. 6D.

FIGS. 7A-7C show schematics of exemplary methods for forming sutures and other valve components configured to reduce pannus growth in one aspect.

FIGS. 8A-8B show an exemplary implantable prosthetic heart valve in one aspect. FIG. 8A shows a front view. FIG. 8B shows a side view of the prosthetic heart valve of FIG. 8A with the outer skirt removed to show an inner skirt and valvular structure mounted on the frame.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### DEFINITIONS

As used in this application and in the claims, the singular forms “a,” “an,” and “the” include the plural forms unless the context clearly dictates otherwise. Thus, for example, reference to a “suture” includes aspects having two or more such sutures unless the context clearly indicates otherwise.

It is also to be understood that the terminology used herein is for the purpose of describing particular aspects only and is not intended to be limiting. As used in the specification and in the claims, the term “comprising” can include the aspects “consisting of” and “consisting essentially of.” Additionally, the term “includes” means “comprises.”

For the terms “for example” and “such as” and grammatical equivalences thereof, the phrase “and without limitation” is understood to follow unless explicitly stated otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint and independently of the other endpoint. Unless stated otherwise, the term “about” means within 5% (e.g., within 2% or 1%) of the particular value modified by the term “about.” It is further understood that if the term “about 1” is disclosed, then also term “1” is disclosed.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments there between. This applies regardless of the breadth of the range.

As used herein, the terms “optional” or “optionally” mean that the subsequently described event or circumstance can or cannot occur and that the description includes instances where said event or circumstance occurs and instances where it does not.

Further, the terms “coupled” and “associated” generally mean electrically, electromagnetically, and/or physically (e.g., mechanically or chemically) coupled or linked and do not exclude the presence of intermediate elements between the coupled or associated items.

It will be understood that when an element is referred to as being “connected” or “coupled” to another element, it can be directly connected or coupled to the other element, or intervening elements can be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

It will be understood that although the terms “first,” “second,” etc., can be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example aspects.

Spatially relative terms, such as,” “beneath,” “below,” “lower,” “above,” “upper,” “upward,” “downward,” “top,” “bottom,” and the like, can be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device can be otherwise oriented (rotated 90 degrees or at other orientations), and the spatially relative descriptors used herein are interpreted accordingly.

Terms such as “proximal,” “distal,” “radially outward,” “radially inward,” “outer,” “inner,” and “side” describe the orientation and/or location of portions of the components or elements within a consistent but arbitrary frame of reference which is made clear by reference to the text and the associated drawings describing the components or elements under discussion. Such terminology can include the words specifically mentioned above, derivatives thereof, and words of similar import.

As used herein, the term “and/or” includes any and all combinations of one or more of the associated listed items. It is also understood that the term “and/or” includes where one or another of the associated listed items is present, and the aspects where both of the associated listed items are present or any combinations of the associated listed items are present.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

The terms “fiber” or “filament” can be used interchangeably.

The terms “fiber” (or “filament”) and “material comprising a plurality of fibers (filaments)” are used herein according to their broad and ordinary meanings and can refer to any type of natural or synthetic substance or material that is significantly longer than it is wide, including any elongate or relatively fine, slender, and/or threadlike piece, filament, cord, yarn, plie, strand, line, string, or portion thereof. Furthermore, “fiber” or “material comprising a plurality of fibers” can refer to a single filament or collectively to a plurality of filaments. Examples of material comprising a plurality of fibers in accordance with aspects of the present disclosure include, but are not limited to, any type of cloth, fabric, or textile. It is understood that in certain unlimiting aspects, the term “material comprising a plurality of fibers” can refer to cloth, fabric, textile, or interlocking-fiber material that can cover or form certain features of the disclosed devices.

The term “fiber,” as used herein, includes fibers of extreme or indefinite length (e.g., filaments) and fibers of short length (e.g., staple fibers). It is further understood that the filaments can be monofilaments, multifilaments, braided filaments, unbraided filaments, textured filaments, untextured filaments, and the like.

As used herein, the term “substantially” means that the subsequently described event or circumstance completely occurs or that the subsequently described event or circumstance generally, typically, or approximately occurs.

Still further, the term “substantially” can, in some aspects, refer to at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

As used herein, the term “substantially,” in, for example, the context “substantially identical” or “substantially similar,” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

Although the operations of exemplary aspects of the disclosed method can be described in a particular, sequential order for convenient presentation, it should be understood that disclosed aspects can encompass an order of operations other than the particular, sequential order disclosed. For example, operations described sequentially can, in some cases, be rearranged or performed concurrently. Further, descriptions and disclosures provided in association with one particular aspect are not limited to that aspect and can be applied to any aspect disclosed.

Moreover, for the sake of simplicity, the attached figures cannot show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

### IMPLANTABLE PROSTHETIC DEVICE

One of the challenges in the field of implantable prosthetic devices that affect the device’s longevity and its general performance is uncontrollable tissue and pannus growth on an inner (luminal) surface of the device. The pannus tissue growth is a non-immune inflammatory reaction of the body to the implanted prosthesis. The starting point for such growth can be any blood turbulence-inducing surfaces. Often, the pannus growth is initiated in the suture area of the valve and propagates from there. The currently available solution to avoid this problem is to administer dual antiplatelet therapy (DAPT) to the patients. However, this solution requires patients to take 2 types of antiplatelet medicines for a prolonged time, and it only slows down the pannus growth rate but does not eliminate it. In extreme cases, a valve replacement is required, but it may not be applicable for all pannus based malfunctioned valves.

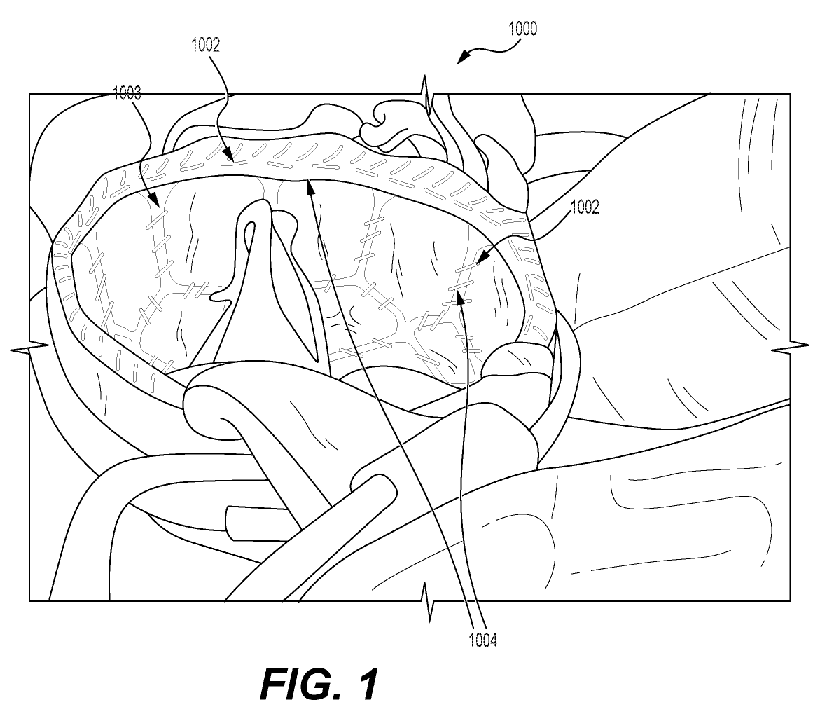

FIG. 1 shows an exemplary valve 1000 positioned in a patient’s native anatomy. It is understood that the native anatomy can include, for example, and without limitations, the valvular locations, such as the aortic, mitral, tricuspid, and pulmonic valves, and their surrounding locations. Exemplary sutures 1002 are used to attach various portions of the valve to the frame 1003. In certain unlimiting aspects, the pannus growth can be observed around areas marked as 1004. It is understood that the pannus growth can be initiated in other portions of the inner surface of the valve.

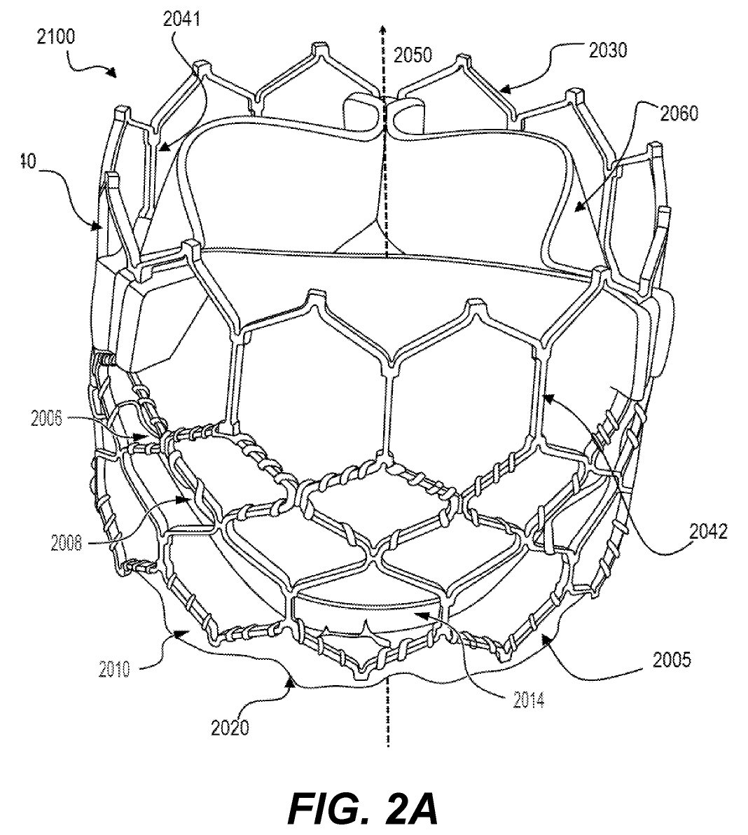

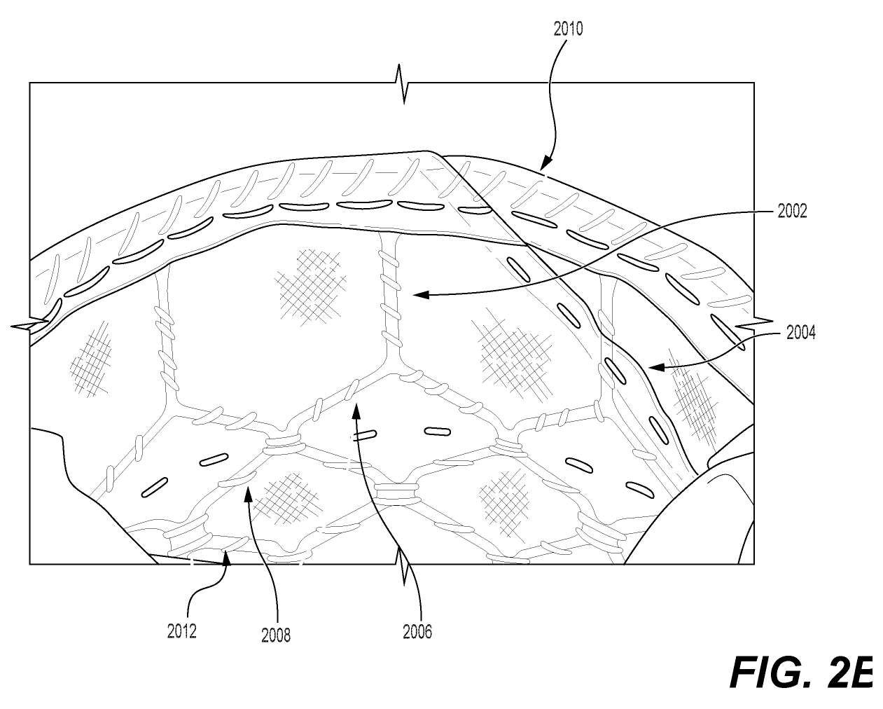

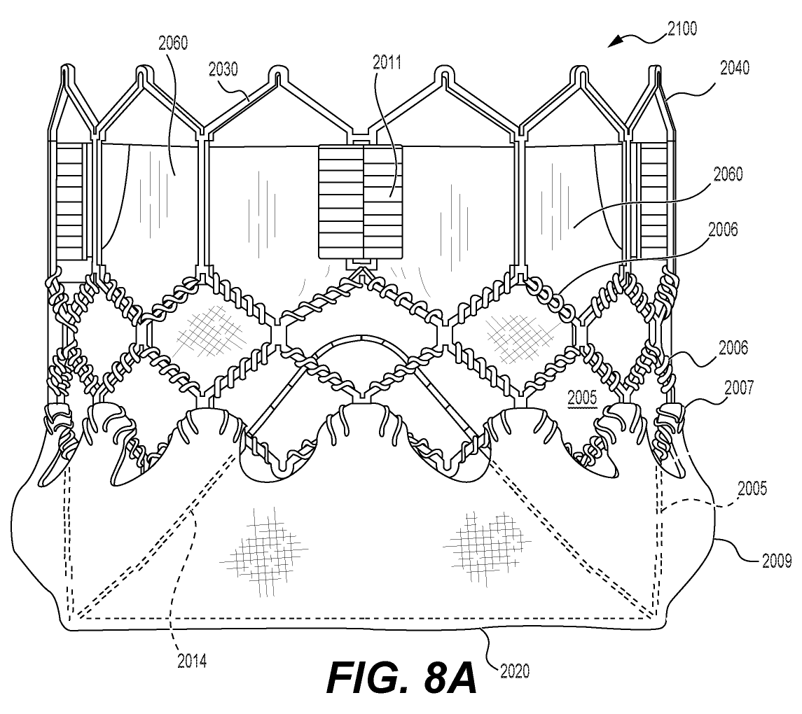

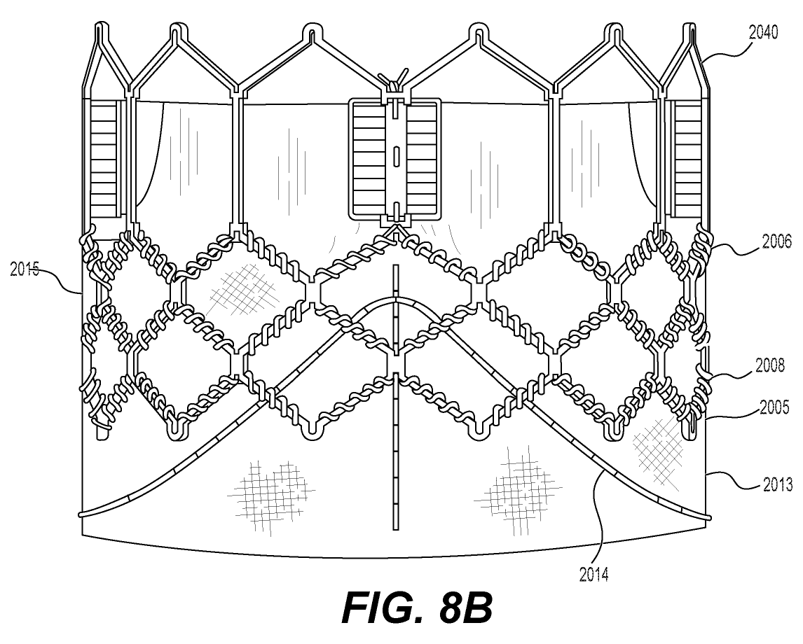

The present disclosure is directed to the implantable prosthetic valves having one or more sutures that are configured to reduce the formation of pannus and other tissue growth. A partial schematic of an exemplary implantable prosthetic device 2100 is shown in FIG. 2A. More detailed schematics of the exemplary valves are shown in FIGS. 8A-8B and will be discussed herein.

In one aspect disclosed herein is an implantable prosthetic valve 2100, comprising: an annular frame 2040 having an inner surface 2041 (luminal) and an outer surface 2042 (abluminal), wherein the frame has an inflow end 2020 and an outflow end 2030, and a central longitudinal axis 2050 extending from the inflow end to the outflow end. In still further aspects, the implantable prosthetic valve also comprises an inner skirt 2005 (partially shown in FIG. 2A) having an inner surface and an outer surface and positioned along the inner surface of the frame. In still further aspects, the valve can optionally comprise a leaflet structure 2060 comprising one or more leaflets, having an inner surface and an outer surface, and positioned at the inner surface of the annular frame. In still further aspects, the valve can optionally comprise an outer skirt. The outer skirt 2009 as shown, for example, in FIG. 8A has an inner surface and an outer surface and is positioned at the outer surface of the annular frame 2040. The outer skirt can also be attached to the frame with one or more sutures 2007.

In still further aspects, the implantable medical device disclosed herein comprises one or more sutures 2002, 2004, 2006, 2008, 2010, 2012, and 2014 as for example shown in FIGS. 2A and 2B. The sutures shown in FIGS. 2A and 2B run along various portions of the device. In still further aspects, the sutures can be used to attach at least a portion of the inner skirt and/or at least a portion of the leaflet structure and/or at least a portion of the outer skirt, if present, to at least a frame of an implantable prosthetic valve. In yet still further aspects, the sutures can be used to attach various components of the valve together. For example, sutures 2002 run along vertical struts of the frame. Sutures 2004 run along the skirt joint. Sutures 2006, 2008, 2010, and 2012 run along various rows of struts in the frame, while suture 2014 runs along the leaflet 2060 cusp.

In still further aspects, the one or more sutures are configured to attach at least a portion of the inner skirt, at least a portion of the leaflet structure and/or at least a portion of the outer skirt, if present, to at least the annular frame and wherein the one or more sutures are configured to substantially reduce pannus formation and/or uncontrolled tissue ingrowth along the implantable prosthetic valve. It is understood that in some aspects, the at least portions described above are on the inner surfaces of the implantable prosthetic device. In still further aspects, the one or more sutures disclosed herein are configured to substantially reduce pannus formation and/or uncontrolled tissue ingrowth along the inner surface of the implantable prosthetic valve. It is further understood that in some aspects, having pannus limiting materials on the outer surfaces, or at least in some portions of the outer surfaces of the implantable prosthetic device, can be undesirable.

In still further aspects, the one or more sutures can have any desirable form and shape. In certain aspects, the one or more sutures can have a substantially cylindrical form. It is understood that in some aspects, the cylindrical sutures can be monofilament. Yet, in other aspects, the cylindrical sutures can be multifilament. In still further aspects, if the sutures are multifilament, such filaments can be braided, textured, piled, twisted, or without twist but in a bundle (Chainette yarns).

In certain aspects, the one or more sutures have a substantially flattened form. In such exemplary and unlimiting aspects, the flattened sutures can be present as a tape. Yet in other aspects and as disclosed below, the original piled, braided, and/or twisted sutures can be flattened by any methods known in the art, for example, and without limitations by treatment with heat. In still further aspects, the flattened sutures can have a planar shape. In still further aspects, the flattened sutures are substantially free of sharp edges. Yet, in other aspects, the flattened sutures can have substantially curved edges. Yet, in other aspects, the flattened sutures can have any desired shape and/or edge shape.

In still further aspects, at least a portion of a surface of the one or more sutures has a coating comprising a pannus limiting material. In still further aspects, a substantially entire surface of the one or more sutures has the coating comprising the pannus limiting material. It is understood that in such aspects, the pannus limiting material can comprise thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

In still further aspects, any known in the art thermoplastic polyurethanes can be used to form a coating. In certain aspects, the thermoplastic polyurethanes can be modified. For example, and without limitations, the thermoplastic polyurethane can be modified to include fluorine- or silicone-containing moieties incorporated within a polymer structural backbone. It is understood that the phrase “incorporated within a polymer structural backbone” includes both the moieties present in the main chain as well as moieties branched out of the main chain. In still further aspects, the modified thermoplastic polyurethane comprises a mixture of thermoplastic polyurethane and a compound having fluorine-containing moieties and/or silicone.

In still further aspects, any inorganic backbone polymers having the desired properties can be used. In certain aspects, the modified inorganic backbone polymers can be modified with halogen-containing groups, phosphorus-containing groups, nitrogen-containing groups, or a combination thereof. In certain aspects, the modified inorganic backbone polymers can comprise polyphosphazenes. It is understood that halogen-containing groups can comprise groups containing fluorine, chlorine, bromine, iodine, or any combination thereof.

In still further aspects, the polyethylenes comprise ultra-high-molecular-weight-polyethylene (UHMWPE), low-density polyethylene (LDPE), high density polyethylene (HDPE), or any combination thereof.

In still further aspects, the coating is applied to the one or more sutures prior to the attachment of the at least a portion of the inner skirt and/or the at least a portion of the leaflet structure and/or the at least a portion of the outer skirt if present, to at least the annular frame. It is understood that the issue of uncontrollable pannus growth is especially problematic on the inner surfaces of the implantable device. The portions of the outer skirt that contact the natural anatomy of a patient body in some aspects are desired to be configured to allow tissue ingrowth. Thus, it is possible only to use the one or more sutures disclosed herein on the portions of the outer skirt where the tissue growth is undesirable. Similarly, the one or more sutures disclosed herein can be used only on the portions of the leaflet structures where the tissue ingrowth is undesirable. Yet in still further aspects, using the disclosed herein one or more sutures to create a desirable pattern on any of the disclosed components that would combine the locations were the tissue ingrowth is desired with the locations fee of tissue ingrowth as needed.

While in other aspects, some portions of the various components of the valve, such as at least a portion of the inner skirt, and/or at least a portion of the leaflet structure, and/or at least a portion of the outer skirt, if present, can comprise the coating comprising the pannus limiting material. As discussed above, it is understood that the portions of the outer skirt facing the native anatomy are configured to allow tissue ingrowth. The disclosed aspects allow the formation of the valve components having portions with the pannus limiting materials as well as without the pannus limiting materials if desired for the specific application. For example, the outer skirt can have inner and outer skirt apex covers, as desired, to prevent tissue ingrowth. In such aspects, the pannus limiting material, as disclosed herein, can be deposited on apex covers to limit growth transfer from outside to inside, forming a no-growth zone. It is also understood that the aspects disclosed herein relate to the outer skirt and/or leaflet structure that are substantially free of pannus limiting materials.

In yet still further aspects, if the pannus limiting material is desired only in the specific locations, other portions of the inner skirt, outer skirt or leaflet valve can be masked prior to applying the pannus limiting material to create the desired pattern.

In certain aspects, disclosed herein is an implantable prosthetic valve comprising: an annular frame having an inner surface and an outer surface wherein the frame has an inflow end and an outflow end, and a central longitudinal axis extending from the inflow end to the outflow end; an inner skirt having an inner surface and an outer surface and positioned along the inner surface of the frame; wherein the inner surface of the inner skirt comprises a first pannus limiting material; an optional leaflet structure having an inner surface and an outer surface and positioned at the inner surface of the annular frame; and/or an optional outer skirt having an inner surface and an outer surface and positioned at the outer surface of the annular frame, and one or more sutures, wherein the one or more sutures are configured to attach at least a portion of the inner skirt, at least a portion of the leaflet structure and/or at least a portion of the outer skirt, if present, to at least the annular frame and wherein the one or more sutures are configured to substantially reduce pannus formation and/or uncontrolled tissue ingrowth along the implantable prosthetic valve.

In still further aspects disclosed are implantable devices with the leaflet structure is present. Yet, in other aspects, disclosed herein are implantable devices where the outer skirt is present. In such aspects, the outer skirt is positioned circumferentially on the outer surface of the annular frame. Again, it is understood that if desired on the specific portion of the outer skirt, the pannus limiting material can be disposed. However, if the outer skirt is desired to allow tissue ingrowth, then substantially no pannus limiting material will be disposed on such portions.

In some aspects, the one or more sutures can comprise polyester, polyethylene, ultrahigh molecular weight polyethylene, silicone, polyurethane, polytetrafluoroethylene, polyether ether ketone (PEEK), fluoropolymer, or natural tissue, or a combination thereof.

In still further aspects, the one or more sutures can comprise a multifilament thread. Again, as discussed herein, the multifilaments can be braided, twisted, piled, flat bundle, textured, or any combination thereof. In such aspects, the thread can comprise two or more filaments wherein the filaments have a thickness from about 0.02 mm to about 0.5 mm, including exemplary values of about 0.03 mm, about 0.04 mm, about 0.05 mm, about 0.06 mm, about 0.07 mm, about 0.1 mm, about 0.12 mm, about 0.15 mm, about 0.17 mm, about 0.2 mm, about 0.22 mm, about 0.25 mm, about 0.27 mm, about 0.3 mm, about 0.32 mm, about 0.35 mm, about 0.37 mm, about 0.4 mm, about 0.42 mm, about 0.45 mm, and about 0.47 mm. It is understood that the thickness can have any value between any two foregoing values or fall within a range formed by any two foregoing values. For example, and without limitations, the thickness can be about 0.05 mm to about 0.5 mm, about 0.1 mm to about 0.5 mm, about 0.2 mm to about 0.5 mm, about 0.3 mm to about 0.5 mm, about 0.02 mm to about 0.4 mm, about 0.02 mm to about 0.3 mm, about 0.02 mm to about 0.2 mm, about 0.02 mm to about 0.1 mm, about 0.02 mm to about 0.05 mm, and so on.

Yet in still further aspects, the sutures can have a size of about 10-0 to about 4, including exemplary values of about 9-0, about 8-0, about 7-0, about 6-0, about 5-0, about 4-0, about 3-0, about 2-0, about 0, about 1, about 2, and about 3 based on USP (United States Pharmacopeia) units. This sizing can be converted to a metric size in Gauge No. of about 0.2 to about 6, including exemplary values of about 0.3, about 0.4, about 0.5, about 0.7, about 1, about 1.5, about 2, about 3, about 3.5, about 4, and about 5. Yet in still further aspects, the sutures can have an average diameter of about 0.02 mm to about 0.7 mm, including exemplary values of about 0.03 mm, about 0.04 mm, about 0.05 mm, about 0.06 mm, about 0.07 mm, about 0.1 mm, about 0.12 mm, about 0.15 mm, about 0.17 mm, about 0.2 mm, about 0.22 mm, about 0.25 mm, about 0.27 mm, about 0.3 mm, about 0.32 mm, about 0.35 mm, about 0.37 mm, about 0.4 mm, about 0.42 mm, about 0.45 mm, about 0.47 mm, about 0.5 mm, about 0.52 mm, about 0.55 mm, about 0.57 mm, about 0.6 mm, about 0.62 mm, about 0.65 mm, and about 0.67 mm. It is understood that the sutures can have any average diameter that falls between any two foregoing values or falls within a range formed by any two foregoing values. For example, and without limitations, the sutures can have an average diameter of about 0.05 mm to about 0.7 mm, about 0.1 mm to about 0.7 mm, about 0.2 mm to about 0.7 mm, about 0.3 mm to about 0.7 mm, about 0.5 mm to about 0.7 mm, about 0.02 mm to about 0.5 mm, about 0.02 mm to about 0.4 mm, about 0.02 mm to about 0.3 mm, about 0.02 mm to about 0.2 mm, about 0.02 mm to about 0.1 mm, about 0.02 mm to about 0.05 mm, and so on.

It is further understood that each of the filaments in the thread can have the same thickness or be different. It is further understood that the overall roughness of the sutures can be dependent on the filament diameter. It is understood that the roughness is measured on the sutures surfaces.

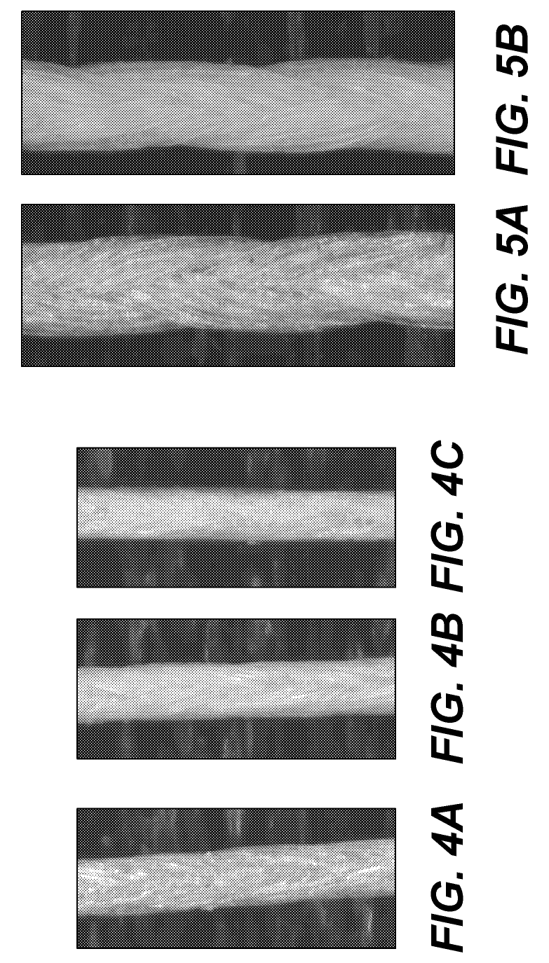

Exemplary sutures are shown in FIGS. 4A–4C, 5A–5B, and 6A–6E. For example, FIG. 4A shows a PET-based suture that has a cylindrical form and does not have any coating disposed on its surface. It can be seen that the suture is a suture’s surface has a substantial roughness that can affect its surface area. FIG. 4B shows an application of the disclosed herein pannus limiting materials substantially on all surfaces of the PET-based suture. It can be seen that the coating covers the suture and reduces the surface area by reducing the roughness. FIG. 4C shows a suture that was first scoured and then coated with any of the disclosed materials herein. FIGS. 5A and 5B show a UHMWPE-based suture without any treatment and coated with the disclosed herein materials, respectively. The changes in the surface roughness are also observed.

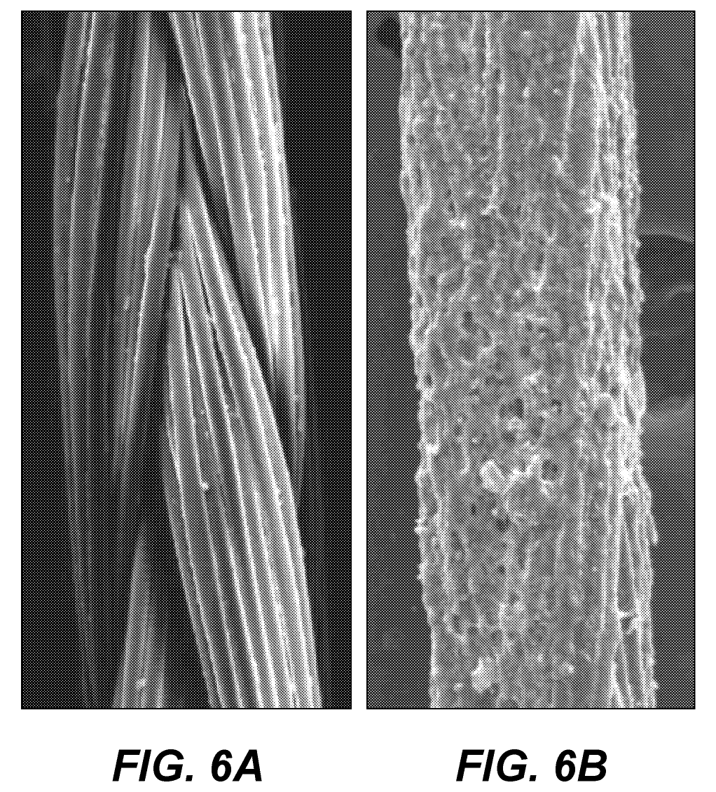

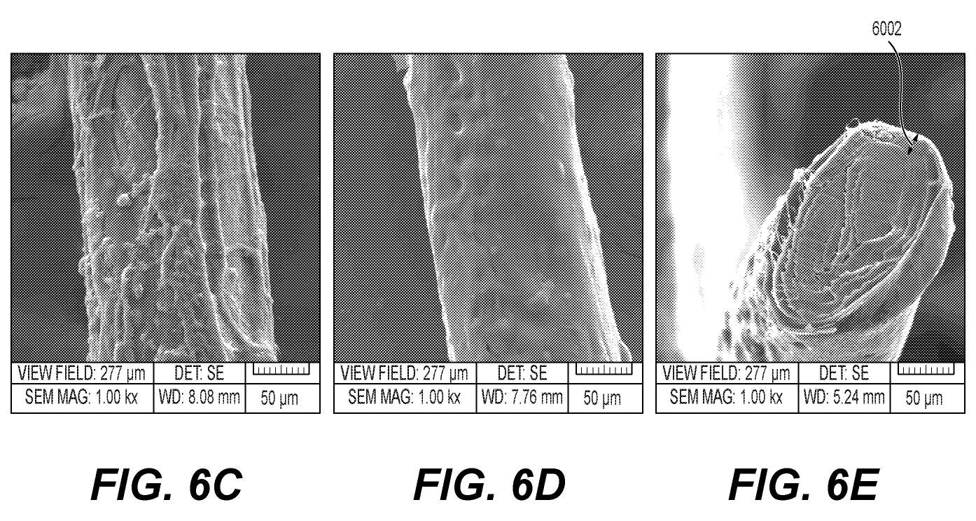

FIG. 6A–6E show SEM images of such exemplary sutures. For example, FIG. 6A shows untreated sutures. FIG. 6B–6C show suture with the disclosed herein coating, while FIG. 6D shows the suture with the coating that was smoothed and heat treated to further reduce the surface area of the suture and reduce its roughness. FIG. 6E shows a cross-section of the coated suture with a coating thickness 6002.

In still further aspects, the at least a portion of the inner skirt, and/or the at least a portion of the leaflet structure, and/or the at least a portion of the outer skirt, if present, and the one or more sutures are substantially free of roughness higher than about 0.5 μm, about 1 μm, 0.5 μm, about 0.1 μm, about 90 nm, about 50 nm, or about 30 nm. Without wishing to be bound by any theory, since tissue cells are about 10 to 15 μm and can also reach dimensions of about 40 μm, it is assumed that any surface roughness below 5 μm would not be detected by the cells and cause cell growth.

In yet still further aspects, surfaces of the one or more sutures are substantially free of a turbulence-inducing surface topography causing a pannus formation. In still further aspects, the at least a portion of the inner skirt is substantially free of a turbulence-inducing surface topography causing a pannus formation. In yet still, further aspects, wherein the at least a portion of the leaflet structure is substantially free of a turbulence-inducing surface topography causing a pannus formation. In yet further aspects, wherein the at least a portion of the outer skirt is substantially free of a turbulence-inducing surface topography causing a pannus formation.

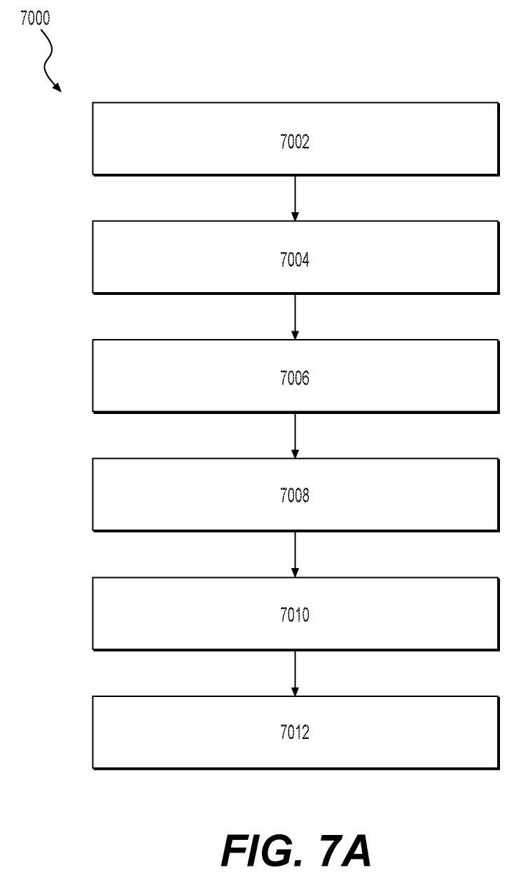

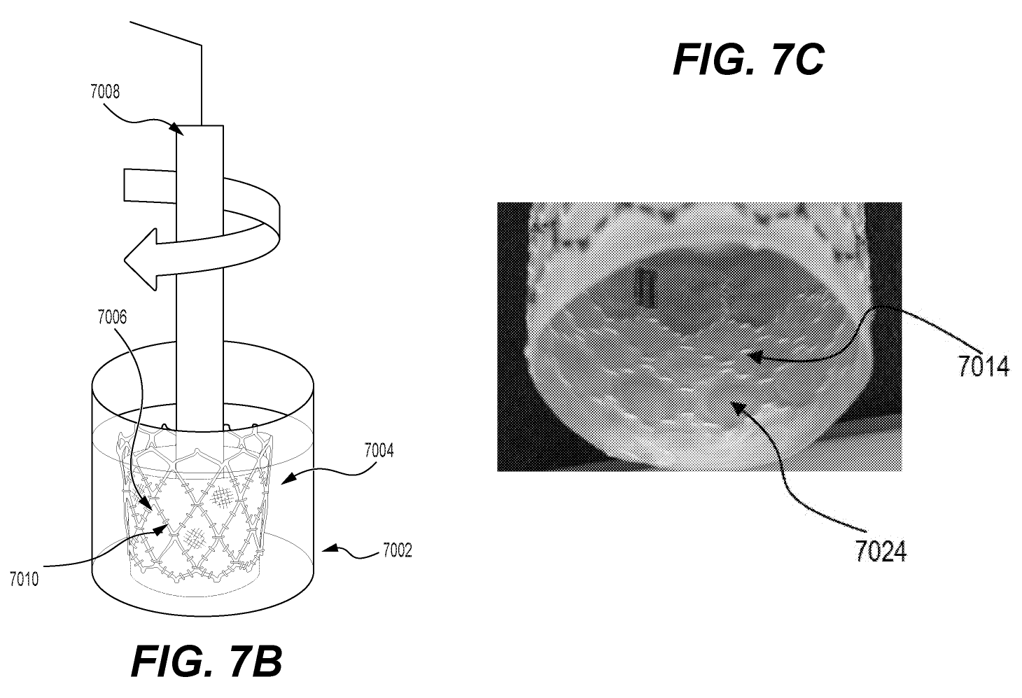

In further aspects, the sutures and the inner surface (and the at least a portion of the outer surface) of the inner skirt form a smooth profile with the sutures used to attach it to at least the frame. It is understood that in such aspects, the overall topography of the inner surface of the inner skirt and the sutures is free of profound edges. In still further aspects, the sutures and the surfaces (at least portions of the inner surface and at least portions of the outer surface) of the inner skirt are configured to form a smooth profile that is substantially free of “pockets” or “trenches” formed between the suture and the surfaces of the inner skirt. An exemplary photograph of such a profile can be seen in FIG. 7C, where the sutures 7014 and the inner skirt 7024 form a smooth profile. It is further understood that the sutures and at least some portions of the leaflet structures and at least some portions of the surfaces of the outer skirt, if present, form similar profiles to the sutures and the surfaces of the inner skirt.

In still further aspects, the coating disclosed herein can have a thickness of about 50 nm to about 150 μm, including exemplary values of about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, and about 145 μm. In still further aspects, the thickness of the coating can have any value between any two foregoing values or fall within any range formed by any two foregoing values. For example, and without limitations, the thickness of the coating can be about 50 nm to about 100 μm, about 50 nm to about 50 μm, about 50 nm to about 10 μm, about 50 nm to about 1 μm, about 50 nm to about 900 nm, about 50 nm to about 500 nm, about 50 nm to about 200 nm, about 50 nm to about 100 nm, about 100 nm to about 150 μm, about 200 nm to about 150 μm, about 500 nm to about 150 μm, about 1 μm to about 150 μm, about 10 μm to about 150 μm, about 50 μm to about 150 μm, about 100 μm to about 150 μm, and so on.

In still further aspects, the implantable prosthetic valve disclosed herein is radially collapsible to a collapsed configuration and radially expandable to an expanded configuration. Exemplary implantable prosthetic valves are disclosed in FIGS. 2A, 8A and 8B.

For example, FIG. 8A shows side plan views, respectively, of an exemplary prosthetic heart valve 2100 having an inflow end 2020 and an outflow end 2030, according to one aspect. For example, prosthetic heart valves 2100 are described in U.S. Patent No. 10,463,484, titled “Prosthetic Heart Valve Having Leaflet Inflow Below Frame,” which is incorporated herein by reference. The illustrated prosthetic valve 2100 is adapted to be implanted in the native aortic annulus, although in other aspects, it can be adapted to be implanted in the other native annuluses of the heart (the mitral valve, pulmonary valve, and tricuspid valve). The prosthetic valve 2100 can have one or more of the following components: a frame, 2040, a leaflet (or valvular) structure 2060, and an inner skirt 2005. The valve 2100 can also include an outer skirt 2009.

In certain aspects, the prosthetic valve 2100 can comprise a leaflet structure 2060. In certain aspects, the leaflet structure can comprise one or more leaflets, each of which can be arranged to collapse in a tricuspid arrangement. The certain aspects, an edge of the leaflet structure 2060 can have an undulating, curved scalloped shape. Moreover, by virtue of the scalloped shape, folds, and ripples at the belly of each leaflet, which can cause early calcification in those areas, can be eliminated or at least minimized. Leaflets can have various other shapes and/or configurations in different aspects. It is understood, however, that the leaflets of the leaflet structure need not have a V-shaped or scalloped inflow edge, and instead, each leaflet can have a square or rectangular shape defining a straight inflow edge.

As shown in FIGS. 2A, 8A, and 8B, the leaflets 2060 can be secured with the disclosed herein one or more sutures 2014 to the frame 2040. The frame 2040 can be made of any of various suitable plastically-expandable materials (e.g., stainless steel, cobalt chromium, etc.) or self-expanding materials (e.g., nitinol) as is known in the art. When constructed of a plastically-expandable material, the frame 2040 can be crimped to a radially compressed state on a delivery catheter and then expanded inside a patient by an inflatable balloon or equivalent expansion mechanism. When constructed of a self-expandable material, the frame 2040 can be crimped to a radially compressed state and restrained in the compressed state by insertion into a sheath or equivalent mechanism of a delivery catheter. Once inside the body, the valve can be advanced from the delivery sheath, which allows the valve to expand to its functional size. Suitable plastically-expandable materials that can be used to form the frame 2040 include, without limitation, stainless steel, a nickel-based alloy (e.g., a cobalt-chromium or a nickel-cobalt-chromium alloy), polymers, or combinations thereof. In particular aspects, frame 2040 can be made of a nickel-cobalt-chromium-molybdenum alloy, such as MP35N™ alloy (tradename of SPS Technologies), which is equivalent to UNS R30035 alloy (covered by ASTM F562-02). MP35N™/UNS R30035 alloy comprises 35% nickel, 35% cobalt, 20% chromium, and 10% molybdenum by weight. In aspects when MP35N is used as the frame material, less material is needed to achieve the same or better performance in radial and crush force resistance, fatigue resistance, and corrosion resistance. Moreover, since less material is required, the crimped profile of the frame can be reduced, thereby providing a lower profile valve assembly for percutaneous delivery to the treatment location in the body.

The frame 2040 can have other configurations or shapes in other aspects.

The leaflets can be sutured together to form the leaflet structure 2060, which can then be secured to the frame 2040. It is understood that when the leaflets are sutured together, each of the leaflets can comprise any of the disclosed herein sutures if desired. However, also disclosed herein are the aspects, when different from the disclosed, one or more sutures are used to secure the leaflet structure within the valve. In still further exemplary and unlimiting aspects, the leaflets of the leaflet structure 2060 can be secured to one another at their adjacent sides to form commissures 2011 of the leaflet structure. It is understood that sutures disclosed herein can also be used to form such commissures. Yet, in other aspects, sutures different from those disclosed herein sutures can also be used.

In still further exemplary aspects, the one or more leaflets of the leaflet structure 2060 can be attached to the inner skirt 2005, with sutures 2014. The suture 2014 can track the curvature of the bottom edge of the leaflet structure 2060 and are collectively referred to as the scallop line.

The inner skirt 2005 can have a plurality of functions, which can include assisting in securing the leaflet structure 2060 and/or the outer skirt 2009 to the frame 2040 and to assist in forming a good seal between the valve 2100 and the native annulus by blocking the flow of blood below the lower edges of the leaflets. It is understood that any known in the art configuration/construction of the skirt can be used. For example, and without limitation, the configuration/construction of the skirt can comprise a textile that is braided, knitted, woven and/or nonwoven. The inner skirt 2005 can comprise a tough, tear-resistant material such as polyethylene terephthalate (PET), although various other synthetic or natural materials can be used. The thickness of the skirt is desirably less than 6 mils or 0.15 mm, and desirably less than 4 mils or 0.10 mm, and even more desirably about 2 mils or 0.05 mm, and even still more desirably about 1.1 mils or 0.03 mm. In particular aspects, the skirt 2005 can have a variable thickness, for example, the skirt can be thicker at its edges than at its center. In one implementation, the skirt 2005 can comprise a PET skirt having a thickness of about 0.07 mm at its edges and about 0.06 mm at its center. The thinner skirt can provide for better crimping performances while still providing good perivalvular sealing. In still further aspects, the inner skirt 2005 can have any of the disclosed coatings herein. In certain aspects, only an inner surface of the inner skirt 2005 can have the disclosed herein coating. While in other aspects, both surfaces of the inner skirt can have the same coating.

Additionally and/or alternatively, as shown in FIG. 8B, the inner skirt 2005 can include a first portion 2015, also referred to as the supported portion, that extends circumferentially around the central longitudinal axis of the prosthetic valve along an inner surface of the frame 2040 and a second portion, also referred to as the unsupported portion 2013, at the inflow end 2020 of the prosthetic valve 2100. The unsupported portion 2013 is unsupported by the frame 2040. The unsupported portion 2013 can extend circumferentially around the central longitudinal axis of the prosthetic valve to form an unsupported circumference at the inflow end 2020.

In certain aspects and as described herein, the prosthetic valve can also comprise an outer skirt 2009. The outer skirt 2009 can be laser cut or otherwise formed from a strong, durable piece of material, such as woven PET, although other synthetic or natural materials can be used. The outer skirt 2009 can be secured to the lower edge of the inner skirt 2005 at the inflow end of the prosthetic valve, such as by any of the disclosed herein sutures. However, other methods of securing the outer skirt are also available, for example, welding and/or an adhesive and the like. In particular aspects, the lower edge of the outer skirt 2009 can be tightly sutured or otherwise secured (e.g., by welding or an adhesive) to the inner skirt 2005. In certain aspects, only the desired portions of the outer skirt can have the disclosed herein pannus limiting materials. In certain aspects, the outer surface of the outer skirt is configured to allow tissue ingrowth. If no tissue growth zone is desired, the disclosed coatings or sutures can be used to achieve this goal.

The absence of metal components of the frame or other rigid members along the inflow end portion of the prosthetic valve advantageously reduces mechanical compression of the native valve annulus (e.g., the aortic annulus) and the left ventricular outflow tract (when implanted in the aortic position), thus reducing the risk of trauma to the surrounding tissue.

Any described herein sutures can be present in a kit. In some aspect, the kit can comprise any of the disclosed herein one or more sutures configured to attach an inner skirt and/or leaflet structure and/or outer skirt to a frame of an implantable prosthetic valve, wherein the one or more sutures are configured to substantially reduce pannus formation and/or uncontrolled tissue ingrowth along the implantable prosthetic valve.

In still further aspects, the kits can comprise the one or more sutures having a substantially cylindrical form, a substantially flattened form, or a combination thereof. In still further aspects, the kits can comprise the one or more sutures, wherein at least a portion of a surface of the one or more sutures has a coating comprising a pannus limiting material. Yet in other aspects, a substantially entire surface of the one or more sutures has the coating comprising the pannus limiting material.

As disclosed herein, wherein the pannus limiting material can comprise any of the disclosed above thermoplastic polyurethanes, modified thermoplastic polyurethanes, polycarbonate urethane, polyether urethane, polyester urethanes, fluoropolymers, fluoro-oligomers, polyethylenes, modified inorganic backbone polymers, polyphospazenes, polyacrylates, polysiloxanes, polyesters, any copolymers thereof, or any combination thereof.

In still further aspects, the one or more sutures present in the kit can comprise a polyester, polyethylene, ultrahigh molecular weight polyethylene, silicone, polyurethane, polytetrafluoroethylene, polyether ether ketone (PEEK), fluoropolymer, or natural tissue, or a combination thereof.

It is understood that these kits can be used in any known application where the coupling of two or more members is required. For example, and without limitation, the kits comprising the disclosed one or more sutures can be used to couple or attach a fabric or a tissue to a frame of a prosthetic valve, or to each other, or to any other desired components. In still further aspects, the one or more sutures present in the disclosed herein kits are configured to couple various components of the article.

### METHODS

The aspects of the present disclosure also relate to methods of making a prosthetic valve comprising attaching at least a portion of an inner skirt to at least an annular frame with one or more sutures such that an outer surface of the inner skirt is at least partially attached to an inner surface of the annular frame, wherein the one or more sutures are configured to substantially reduce pannus formation along the implantable prosthetic valve, and wherein an inner surface of the inner skirt faces an inner space formed by the annular frame. In still further aspects, and as shown above, the annular frame has an inflow end and an outflow end, and a central longitudinal axis extending from the inflow end to the outflow end. In certain aspects, any of the disclosed herein one or more sutures can be utilized. In some aspects, the one or more sutures are configured to substantially reduce pannus formation along the implantable prosthetic valve.

In still further aspects, the method of making the prosthetic valve comprises attaching at least a portion of a leaflet structure having an inner surface and an outer surface with the one or more sutures to at least the annular frame. While in yet still further aspects, the method can comprise attaching at least a portion of an outer skirt with the one or more sutures to at least the annular frame.

As disclosed above, the one or more sutures can be provided in a substantially cylindrical form or in a flattened form. In still further aspects, the suture can have a portion that has a flattened form and a portion that has a substantially cylindrical form. In certain aspects, the sutures can be provided as tapes. Yet, in other aspects, the substantially cylindrical sutures can be flattened to form the flattened sutures.

In certain aspects, the one or more sutures are flattened prior to attaching the inner skirt to at least the annular frame. Yet in still further aspects, the one or more sutures are flattened prior to attaching the at least a portion of the leaflet structure to at least the annular frame. While in still further aspects, the one or more sutures are flattened prior to attaching the at least a portion of the outer skirt to at least the annular frame.

In still further aspects, the flattening can be done by any known in the art method. For example, in some aspects, the substantially cylindrical sutures can be first treated to substantially remove surface roughness and then flattened. Yet, in other aspects, the substantially cylindrical sutures are flattened without any pretreatment. In certain aspects, the flattening of the one or more sutures comprises passing the one or more sutures between at least two heat presses at a temperature of about 120 °C to about 210 °C, including exemplary values of about 130 °C, about 140 °C, about 150 °C, about 160 °C, about 170 °C, about 180 °C, about 190 °C, and about 200 °C, and including ranges about 120 °C to about 210 °C, about 150 °C to about 210 °C, about 170 °C to about 210 °C, about 120 °C to about 200 °C, about 120 °C to about 150 °C, and a pressure of about 0.5 MPa to about 5 MPa, including exemplary values of about 0.8 MPa, about 1 MPa, about 1.2 MPa, about 1.5 MPa, about 1.8 MPa, about 2 MPa, about 2.2 MPa, about 2.5 MPa, about 2.8 MPa, about 3 MPa, about 3.2 MPa, about 3.5 MPa, about 3.8 MPa, about 4 MPa, about 4.2 MPa, about 4.5 MPa, and about 4.8 MPa, and ranges about 0.5 MPa to about 5 MPa, about 1 MPa to about 5 MPa, ab0ut 2.5 MPa to about 5 MPa, about 0.5 MPa to about 4 MPa, about 0.5 MPa to about 2.5 MPa, and so on.

In one and unlimiting aspects, the one or more sutures are substantially free of surface roughness. In yet still further aspects, the one or more sutures are substantially free of roughness higher than about 10 μm, about 5 μm, about 1 μm, 0.5 μm, about 0.1 μm, about 90 nm, about 50 nm, or about 30 nm.

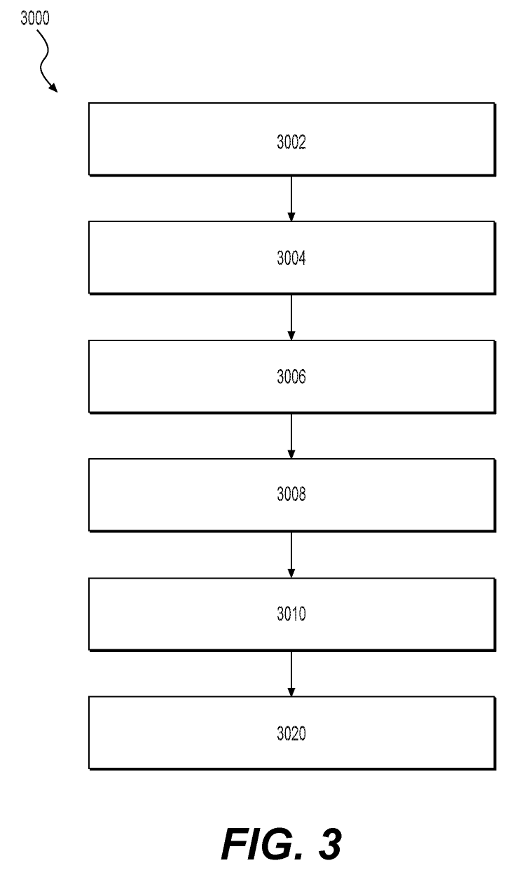

In still further aspects, the method disclosed herein comprises forming a first coating on the one or more sutures, wherein the first coating comprises a pannus limiting material. This method 3000 is schematically shown in FIG. 3. First, the one or more sutures are cleaned 3002. It is understood that the cleaning can be done by any known in the art methods. For example, and without limitations, the sutures can be cleaned with alcohol, such as isopropyl alcohol, and then dried at room temperature. In still further aspects, the step of forming the first coating comprises inserting 3004 the one or more sutures into a first solution comprising a first concentration of a first pannus limiting material for a first predetermined time. In certain aspects, the desired coating can be formed after the inserting step 3004. Yet in other aspects, the method further comprises removing the one or more sutures from the first solution and inserting the one or more sutures into a second solution comprising a second concentration of a second pannus limiting material for a second predetermined time 3006.

In still further aspects, the method can further comprise an additional n number of sequences 3008, wherein each sequence comprises the step of inserting the one or more sutures, for a predetermined time, into asolution comprising a concentration of a pannus limiting material. In such aspects, n can be from 1 to 10, including exemplary values of 2, 3, 4, 5, 6, 7, 8, and 9.

In certain aspects, the first concentration of the first solution is lower than the second concentration of the second solution. However, it is understood that the first concentration can also be greater than the second concentration. In yet still further aspects, the concentration of the pannus limiting material in the each of the n number of sequences is the same or different as the first concentration.

In certain aspects, the first solution, the second solution, and the solution at the each of the n number of sequences can be kept at room temperature or can be heated if desired. In still further aspects, the first predetermined time, the second predetermined time, or the predetermined time in the each of the n number of sequences are the same or different. It is understood that the first predetermined time, the second predetermined time, or the predetermined time in the each of the n number of sequences can be selected to achieve the desired result and can be from about 1 sec to about 5 min, including exemplary values of about 2 sec, about 3 sec, about 4 sec, about 5 sec, about 6 sec, about 7 sec, about 8 sec, about 9 sec, about 10 sec, about 15 sec, about 20 sec, about 25 sec, about 30 sec, about 35 sec, about 40 sec, about 45 sec, about 50 sec, about 55 sec, about 1 min, about 1.5 min, about 2 min, about 2.5 min, about 3 min, about 3.5 min, about 4 min, and about 4.5 min, or ranges about 1 sec to about 5 min, about 1 sec to about 2.5 min, about 1 sec to about 1 min, about 1 sec to about 30 sec, about 15 sec to about 5 min, about 30 sec to about 5 min, about 1 min to about 5 min, and so on In still further aspects, the first predetermined time, the second predetermined time, or the predetermined time in the each of the n number of sequences can have any value between any two foregoing values or can fall within a range formed by any two foregoing values. For example, the first predetermined time, the second predetermined time, or the predetermined time in the each of the n number of sequences can be about 10 sec to about 5 min, about 30 sec to about 5 min, about 1 min to about 5 min, about 2 min to about 5 min, about 1 sec to about 4 min, about 1 sec to about 2 min, about 1 sec to about 1 min, about 1 sec to about 30 sec, and on.

In still further aspects, the first pannus limiting material, the second pannus limiting material, and the pannus limiting material in the each of the n number of sequences are the same or different and are selected from the pannus limiting material comprises thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof. Yet in further aspects, the modified thermoplastic polyurethane comprises thermoplastic polyurethane having fluorine and/or silicone incorporated in a polymer structural backbone. While in other aspects, the modified thermoplastic polyurethane comprises a mixture of thermoplastic polyurethane and a compound having fluorine-containing moieties and/or silicone. In still further aspects, the modified inorganic backbone polymer is modified with halogen-containing groups, phosphorus-containing groups, nitrogen-containing groups, or a combination thereof. While in still further aspects, the polyethylenes comprise wherein the polyethylenes comprise ultra-high-molecular-weight-polyethylene (UHMWPE), low-density polyethylene (LDPE), high density polyethylene (HDPE), or any combination thereof. It is understood that the first solution, the second solution, and/or the solution in the each of the n sequences can also comprise a solvent. Any known in the art solvents can be used. More specifically, the choice of the solvent can be dependent on a choice of pannus limiting material. In some aspects, the solvent can be selected from dimethylacetamide (DMA), tetrahydrofuran (THF), dimethylformamide (DMF), dimethyl sulfide (DMSO), hexafluoro-2-propanol (HFIP), ethyl acetate, butyl acetate, methyl ethyl ketone, acetone, cyclohexanone, or any combination thereof.

In still further aspects, the first concentration, the second concentration, and/or the concentration in the each of the n sequences can be about 1 wt% to about 50 wt%, including exemplary values of about 2 wt%, about 3 wt%, about 4 wt%, about 5 wt%, about 6 wt%, about 7 wt%, about 8 wt%, about 9 wt%, about 10 wt%, about 15 wt%, about 20 wt%, about 25 wt%, about 30 wt%, about 35 wt%, about 40 wt%, and about 45 wt% of the pannus limiting material. In still further aspects, the first concentration, the second concentration, and/or the concentration in the each of the n sequences can have any value between any two foregoing values. In yet still further aspects, the first concentration, the second concentration, and/or the concentration in the each of the n sequences can be about 5 wt% to about 50 wt%, about 10 wt% to about 50 wt%, about 20 wt% to about 50 wt%, about 30 wt% to about 50 wt%, about 1 wt% to about 40 wt%, about 1 wt% to about 30 wt%, about 1 wt% to about 20 wt%, about 1 wt% to about 10 wt%, and so on. In step 3010, the coated sutures are dried to remove any residual solvents, washed with purified water, and dried at room temperature. It is understood that the sutures formed by the methods disclosed herein have a coating that is substantially free of any residual solvents. In still further aspects, in step 3020, the coated sutures can be smoothed, and heat treated. In such aspects, the one or more sutures are smoothed to substantially remove a surface roughness before attaching the at least a portion of the inner skirt, and/or the at least a portion of the leaflet structure, and/or the at least a portion of the outer skirt to at least the annular frame.

In such aspects, a step of smoothing comprises heat treating the one or more sutures having the first coating at a temperature of about 120 °C to about 210 °C, including exemplary values of about 130 °C, about 140 °C, about 150 °C, about 160 °C, about 170 °C, about 180 °C, about 190 °C, and about 200 °C, and a pressure of about 0.5 MPa to about 2.5 MPa, including exemplary values of about 0.8 MPa, about 1 MPa, about 1.2 MPa, about 1.5 MPa, about 1.8 MPa, about 2 MPa, and about 2.2 MPa. In still further aspects, the temperature can have any value that falls within any two foregoing values or can fall in a range formed by any two foregoing values. For example, the temperature can be about 150 °C to about 210 °C, about 170 °C to about 210 °C, about 170 °C to about 210 °C, about 200 °C to about 210 °C, about 120 °C to about 200 °C, about 120 °C to about 170 °C, about 120 °C to about 150 °C, and so on. In still further aspects, the pressure can have any value that falls within any two foregoing values or can fall in a range formed by any two foregoing values. For example, the pressure can be about 0.7 MPa to about 2.5 MPa, about 1 MPa to about 2.5 MPa, about 1.2 MPa to about 2.5 MPa, about 1.5 MPa to about 2.5 MPa, about 1.7 MPa to about 2.5 MPa, about 2 MPa to about 2.5 MPa, about 0.5 MPa to about 2 MPa, about 0.5 MPa to about 1.5 MPa, about 0.5 MPa to about 1 MPa, and so on.

In still further aspects, the steps of smoothing the surface can comprise placing the sutures into a cylinder positioned between two heat presses and passing the suture through the cylinder for a number of times (for example, 1–10 times), wherein each pass ensures that the suture is exposed to a desired temperature and pressure for the desired time, for example for any time of about 1 sec to about 5 min, including exemplary values of about 2 sec, about 3 sec, about 4 sec, about 5 sec, about 6 sec, about 7 sec, about 8 sec, about 9 sec, about 10 sec, about 15 sec, about 20 sec, about 25 sec, about 30 sec, about 35 sec, about 40 sec, about 45 sec, about 50 sec, about 55 sec, about 1 min, about 1.5 min, about 2 min, about 2.5 min, about 3 min, about 3.5 min, about 4 min, and about 4.5 min. In still further aspects, the desired time can have any value between any two foregoing values or can fall within a range formed by any two foregoing values. For example, the desired time can be about 10 sec to about 5 min, about 30 sec to about 5 min, about 1 min to about 5 min, about 2 min to about 5 min, about 1 sec to about 4 min, about 1 sec to about 2 min, about 1 sec to about 1 min, about 1 sec to about 30 sec, and on.

In still further aspects, the first coating formed on the one or more sutures is substantially stable. It is understood that it is desirable that the first coating produces a substantially not particular material that can be dislodged into the native anatomy of the patient. Yet, in other aspects, it is understood that if any particulates are formed, their concentration and size are substantially below the concentration or size that could cause a stroke or embolism-related risk to a patient. In still further aspects, it is understood that the smoothing of the first coating, as disclosed herein, is also aimed to substantially decrease or even fully eliminate the formation of any particulates. In still further aspects, to substantially decrease the formation of particulates, annealing of the first coating can be performed. In such exemplary and unlimiting aspects, the annealing at a temperature of about 60 °C to about 150 °C, including exemplary values of about 70 °C, about 80 °C, about 90 °C, about 100 °C, about 110 °C, about 120 °C, about 130 °C, about 140 °C for a time period of about 3 min to about 15 min, including exemplary values of about 4 min, about 5 min, about 6 min, about 7 min, about 8 min, about 10 min, about 11 min, about 12 min, about 13 min, and about 14 min. In still further aspects, the temperature can have any value that falls within any two foregoing values or can fall in a range formed by any two foregoing values. For example, the temperature can be about 60 °C to about 150 °C, about 70 °C to about 150 °C, about 80 °C to about 150 °C, about 100 °C to about 150 °C, about 60 °C to about 100 °C, about 60 °C to about 70 °C, and so on. In still further aspects, the time can have any value that falls within any two foregoing values or can fall in a range formed by any two foregoing values. For example, the time can be about 5 min to about 15 min, about 7 min to about 15 min, about 10 min to about 15 min, about 5 min to about 12 min, about 5 min to about 10 min, about 5 min to about 7 min, and so on.

In addition, or in the alternative, the method of forming the implantable prosthetic valve can comprise immersing the annular frame with at least a portion of the attached inner skirt into a mixture comprising a pannus limiting material present in a predetermined concentration for a predetermined time to form a second coating. The steps of this method 7000 are schematically shown in FIG. 7A and 7B. For example, the inner skirt is first attached 7002 to at least the annular frame with the one or more sutures. In one aspect, the sutures are not treated prior to the step of the attachment. In other aspects, the sutures are treated to form the first coating having the pannus limiting material. The skirt and the sutures are cleaned and dried in step 7004 and then immersed 7006 into the mixture for at least one time.

In still further aspects, the method can comprise an m number of sequences 7008, wherein each sequence comprises the step of immersing, for a further predetermined time, into a further mixture having a further pannus limiting material in a further predetermined concentration, wherein m is from 1 to 10 (including 2, 3, 4, 5, 6, 7, 8, and 9), and wherein at least one applies: the further pannus limiting materials in the each of the m sequences are the same or different; the further predetermined concentrations in the each of the m sequences are the same or different; and/or; the further predetermined times in the each of the m sequences are the same or different.

Again, it is understood that any of the disclosed above pannus limiting materials can be utilized. In yet other aspects, the mixture can comprise any of the disclosed above solvents.

In still other aspects, the further predetermined time in the each of the m sequences can be selected to achieve the desired result and can be from about 1 sec to about 5 min, including exemplary values of about 2 sec, about 3 sec, about 4 sec, about 5 sec, about 6 sec, about 7 sec, about 8 sec, about 9 sec, about 10 sec, about 15 sec, about 20 sec, about 25 sec, about 30 sec, about 35 sec, about 40 sec, about 45 sec, about 50 sec, about 55 sec, about 1 min, about 1.5 min, about 2 min, about 2.5 min, about 3 min, about 3.5 min, about 4 min, and about 4.5 min. In still further aspects, the time can have any value between any two foregoing values, or can fall within a range formed by any two foregoing values. For example, the time can be about 5 sec to about 5 min, about 10 sec to about 5 min, about 30 sec to about 5 min, about 1 min to about 5 min, about 2 min to about 5 min, about 1 sec to about 4 min, about 1 sec to about 2 min, about 1 sec to about 1 min, about 1 sec to about 30 sec, and on.

In still further aspects, the further predetermined concentration in the each of the m sequences can be about 1 wt% to about 50 wt%, including exemplary values of about 2 wt%, about 3 wt%, about 4 wt%, about 5 wt%, about 6 wt%, about 7 wt%, about 8 wt%, about 9 wt%, about 10 wt%, about 15 wt%, about 20 wt%, about 25 wt%, about 30 wt%, about 35 wt%, about 40 wt%, and about 45 wt% of the pannus limiting material. In still further aspects, the concentration can have any value between any two foregoing values or can fall within a range formed by any two foregoing values. For example, the concentration can be about 5 wt% to about 50 wt%, about 10 wt% to about 50 wt%, about 20 wt% to about 50 wt%, about 30 wt% to about 50 wt%, about 1 wt% to about 40 wt%, about 1 wt% to about 30 wt%, about 1 wt% to about 20 wt%, and so on.

In some aspects, repeated immersion is done sequentially with minimal residence time in between. Yet in other aspects, after each immersing step, the frame with the attached by one or more sutures skirt can be left at least partially to dry before a new immersing step. In 7010, when the desired thickness of the second coating is achieved, the frame with the attached skirt that is attached using one or more sutures is air-dried to ensure the removal of all residual solvents. In step 7012, the frame with the attached that is attached using one or more sutures are thoroughly water-washed and air-dried. Again, it is understood that the second coating formed by the disclosed herein methods is substantially stable. It is understood that it is desirable that the second coating produces a substantially not particular material that can be dislodged into the native anatomy of the patient. Yet, in other aspects, it is understood that if any particulates are formed, their concentration and size are substantially below the concentration or size that could cause a stroke or embolism-related risk to a patient. In still further aspects, to substantially decrease the formation of particulates, annealing of the second coating can be performed. In such exemplary and unlimiting aspects, the annealing of at a temperature of about 60 °C to about 150 °C, including exemplary values of about 70 °C, about 80 °C, about 90 °C, about 100 °C, about 110 °C, about 120 °C, about 130 °C, about 140 °C for a time period of about 3 min to about 15 min, including exemplary values of about 4 min, about 5 min, about 6 min, about 7 min, about 8 min, about 10 min, about 11 min, about 12 min, about 13 min, and about 14 min. In still further aspects, the annealing temperature can have any value that falls within any two foregoing values or can fall in a range formed by any two foregoing values. For example, the annealing temperature can be about 60 °C to about 150 °C, about 70 °C to about 150 °C, about 80 °C to about 150 °C, about 100 °C to about 150 °C, about 60 °C to about 100 °C, about 60 °C to about 70 °C, and so on. In still further aspects, the time can have any value that falls within any two foregoing values or can fall in a range formed by any two foregoing values. For example, the time can be about 5 min to about 15 min, about 7 min to about 15 min, about 10 min to about 15 min, about 5 min to about 12 min, about 5 min to about 10 min, about 5 min to about 7 min, and so on.

The process is schematically shown in FIG. 7B. The frame 7010 with the skirt 7004 attached by one or more sutures 7006 is inserted in a mixture 7002 for the desired time. In some aspects, the frame 7010 with the skirt 7004 attached by one or more sutures 7006 can be rotated by any means 7008 to ensure that the formed second coating is uniform. In certain aspects, the mixture 7002 can be at room temperature. While in other aspects, the mixture 7002 can be heated.

In still further aspects, if at least a portion of the leaflet structure and/or at least a portion of the outer skirt are already attached to at least the annular frame, and the second coating is not desired on these surfaces, the leaflet structure and/or the outer skirt can be masked prior to the step of immersing. Masking can be done to ensure that no material is disposed on the leaflets and/or outer skirt. It is understood that if there are portions where the second coating is desired on the leaflet structure and/or outer skirt, masking can be done appropriately but only concealing the portions where the coating is not desired. Yet, in other aspects, the method can comprise attaching at least a portion of the leaflet structure and/or at least a portion of the outer skirt to at least the annular frame with the one or more sutures after the second coating is formed. In such exemplary and unlimiting aspects, the sutures used to attach at least a portion of the leaflet structure and/or at least a portion of the outer skirt can have a first coating. Yet, in other aspects, the sutures used to attach at least a portion of the leaflet structure and/or at least a portion of the outer skirt can be flattened.

In still further aspects, the one or more sutures are treated according to any methods disclosed herein.

In still further aspects, the first and/or second coatings formed by the disclosed herein methods can have a thickness of about 50 nm to about 150 μm, including exemplary values of about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, and about 145 μm. In still further aspects, the thickness of the first and/or second coatings can have any value between any two foregoing values or fall within any range formed by any two foregoing values. For example, and without limitations, the thickness of the first and/or second coatings can be about 50 nm to about 100 μm, about 50 nm to about 50 μm, about 50 nm to about 10 μm, about 50 nm to about 1 μm, about 50 nm to about 900 nm, about 50 nm to about 500 nm, about 50 nm to about 200 nm, about 50 nm to about 100 nm, about 100 nm to about 150 μm, about 200 nm to about 150 μm, about 500 nm to about 150 μm, about 1 μm to about 150 μm, about 10 μm to about 150 μm, about 50 μm to about 150 μm, about 100 μm to about 150 μm, and so on.

Also disclosed herein is a method for preventing tissue ingrowth, comprising inserting any of the disclosed herein implantable prosthetic valves into a patient’s body.

### EXEMPLARY ASPECTS

EXAMPLE 1: An implantable prosthetic valve comprising: an annular frame having an inner surface and an outer surface wherein the frame has an inflow end and an outflow end, and a central longitudinal axis extending from the inflow end to the outflow end; an inner skirt having an inner surface and an outer surface and positioned along the inner surface of the frame; an optional leaflet structure having an inner surface and an outer surface and attached to the inner surface of the annular frame; and/or an optional outer skirt having an inner surface and an outer surface and positioned at the outer surface of the annular frame, and one or more sutures, wherein the one or more sutures are configured to attach at least a portion of the inner skirt, at least a portion of the leaflet structure and/or at least a portion of the outer skirt, if present, to at least the annular frame, and wherein the one or more sutures are configured to substantially reduce pannus formation and/or uncontrolled tissue ingrowth along the implantable prosthetic valve.

EXAMPLE 2: The implantable prosthetic valve of any examples herein, particularly example 1, wherein the one or more sutures have a substantially cylindrical form.

EXAMPLE 3: The implantable prosthetic valve of any examples herein, particularly example 1, wherein the one or more sutures have a substantially flattened form.

EXAMPLE 4: The implantable prosthetic valve of any examples herein, particularly examples 1–3, wherein at least a portion of a surface of the one or more sutures has a coating comprising a pannus limiting material.

EXAMPLE 5: The implantable prosthetic valve of any examples herein, particularly example 4, wherein a substantially entire surface of the one or more sutures has the coating comprising the pannus limiting material.

EXAMPLE 6:The implantable prosthetic valve of any examples herein, particularly examples 4–5, wherein the pannus limiting material comprises thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

EXAMPLE 7: The implantable prosthetic valve of any examples herein, particularly example 6, wherein the modified thermoplastic polyurethane comprises thermoplastic polyurethane having fluorine and/or silicone incorporated in a polymer structural backbone.

EXAMPLE 8: The implantable prosthetic valve of any examples herein, particularly example 6, wherein the modified thermoplastic polyurethane comprises a mixture of thermoplastic polyurethane and a compound having fluorine-containing moieties and/or silicone.

EXAMPLE 9: The implantable prosthetic valve of any examples herein, particularly example 6, wherein the modified inorganic backbone polymer is modified with halogen-containing groups, phosphorus-containing groups, nitrogen-containing groups, or a combination thereof.

EXAMPLE 10: The implantable prosthetic valve of any examples herein, particularly example 6, wherein the polyethylenes comprise ultra-high-molecular-weight-polyethylene (UHMWPE), low-density polyethylene (LDPE), high density polyethylene (HDPE), or any combination thereof.

EXAMPLE 11: The implantable prosthetic valve of any examples herein, particularly examples 4–10, wherein the coating is applied to the one or more sutures prior to the attachment of the at least a portion of the inner skirt, and/or the at least a portion of the leaflet structure, and/or the at least a portion of the outer skirt, if present, to at least the annular frame.

EXAMPLE 12: The implantable prosthetic valve of any examples herein, particularly examples 4–10, wherein the coating is applied to the one or more sutures after the attachment of the at least a portion of the inner skirt, and/or the at least a portion of the leaflet structure, and/or the at least a portion of the outer skirt, if present, to at least the annular frame.

EXAMPLE 13: The implantable prosthetic valve of any examples herein, particularly example 12, wherein the at least a portion of the inner skirt, and/or the at least a portion of the leaflet structure, and/or the at least a portion of the outer skirt, if present, comprise the coating comprising the pannus limiting material.

EXAMPLE 14: The implantable prosthetic valve of any examples herein, particularly examples 1–13, wherein the at least a portion of the inner skirt, and/or the at least a portion of the leaflet structure, and/or the at least a portion of the outer skirt, if present, and the one or more sutures are substantially free of roughness higher than about 0.5 μm.

EXAMPLE 15: The implantable prosthetic valve of any examples herein, particularly examples 1–14, wherein the at least a portion of the inner skirt, and/or the at least a portion of the leaflet structure, and/or the at least a portion of the outer skirt, if present, and the one or more sutures are substantially free of a turbulence inducing surface topography causing a pannus formation.

EXAMPLE 16: The implantable prosthetic valve of any examples herein, particularly examples 1–15, wherein the at least a portion of the inner skirt, and/or the at least a portion of the leaflet structure, and/or the at least a portion of the outer skirt, if present, and the one or more sutures together form a substantially uniform surface profile.

EXAMPLE 17: The implantable prosthetic valve of any examples herein, particularly examples 4–16, wherein the coating has a thickness of about 50 nm to about 150 μm.

EXAMPLE 18: The implantable prosthetic valve of any examples herein, particularly examples 1–17, wherein the leaflet structure is present.

EXAMPLE 19: The implantable prosthetic valve of any examples herein, particularly examples 1–18, wherein the outer skirt is present and is positioned circumferentially on the outer surface of the annular frame.

EXAMPLE 20: The implantable prosthetic valve of any examples herein, particularly examples 1–19, wherein the implantable prosthetic valve is radially collapsible to a collapsed configuration and radially expandable to an expanded configuration.

EXAMPLE 21: The implantable prosthetic valve of any examples herein, particularly examples 1–20, wherein the one or more sutures comprise polyester, polyethylene, ultrahigh molecular weight polyethylene, silicone, polyurethane, polytetrafluoroethylene, polyether ether ketone (PEEK), fluoropolymer, or natural tissue, or a combination thereof.

EXAMPLE 22: A method of forming an implantable prosthetic valve comprising: attaching at least a portion of an inner skirt to at least an annular frame with one or more sutures such that an outer surface of the inner skirt is at least partially attached to an inner surface of the annular frame, wherein the one or more sutures are configured to substantially reduce pannus formation along the implantable prosthetic valve, and wherein an inner surface of the inner skirt faces an inner space formed by the annular frame; and wherein the annular frame has an inflow end and an outflow end, and a central longitudinal axis extending from the inflow end to the outflow end.

EXAMPLE 23: The method of any examples herein, particularly example 22, comprising attaching at least a portion of a leaflet structure having an inner surface and an outer surface with the one or more sutures to at least the annular frame.

EXAMPLE 24: The method of any examples herein, particularly examples 22 or 23, comprising attaching at least a portion of an outer skirt with the one or more sutures to at least the annular frame.

EXAMPLE 25: The method of any examples herein, particularly examples 22–24, wherein the one or more sutures are flattened prior to attaching the at least a portion of the inner skirt to at least the annular frame.

EXAMPLE 26: The method of any examples herein, particularly examples 23–25, wherein the one or more sutures are flattened prior to attaching the at least a portion of the leaflet structure to at least the annular frame.

EXAMPLE 27: The method of any examples herein, particularly examples 24–26, wherein the one or more sutures are flattened prior to attaching the at least a portion of the outer skirt to at least the annular frame.

EXAMPLE 28: The method of any examples herein, particularly examples 25–27, wherein flattening of the one or more sutures comprises passing the one or more sutures between at least two heat presses at a temperature of about 120 °C to about 210 °C and a pressure of about 0.5 MPa to about 5 MPa.

EXAMPLE 29: The method of any examples herein, particularly examples 22–28, the one or more sutures are substantially free of surface roughness.

EXAMPLE 30: The method of any examples herein, particularly examples 22–29, wherein the one or more sutures have a first coating comprising a pannus limiting material.

EXAMPLE 31: The method of any examples herein, particularly example 30, wherein forming the first coating comprises inserting the one or more sutures into a first solution for a first predetermined time, wherein the first solution comprises a first concentration of a first pannus limiting material.

EXAMPLE 32: The method of any examples herein, particularly example 31, wherein the step forming the first coating further comprises inserting the one or more sutures into a second solution for a second predetermined time, wherein the second solution comprises a second concentration of a second pannus limiting material.

EXAMPLE 33: The method of any examples herein, particularly example 32, wherein the method further comprises an additional n number of sequences, wherein each sequence comprises the step of inserting the one or more sutures, for a predetermined time, into asolution comprising a concentration of a pannus limiting material, and wherein n is from 1 to 10.

EXAMPLE 34: The method of any examples herein, particularly examples 30–33, wherein the first concentration is lower than the second concentration or wherein the first concentration is higher than the second concentration.

EXAMPLE 35: The method of any examples herein, particularly examples 33–34, wherein the first predetermined time, the second predetermined time, or the predetermined time in the each of the n number of sequences are the same or different.

EXAMPLE 36: The method of any examples herein, particularly examples 33–35, wherein the concentration of the pannus limiting material in the each of the n number of sequences is the same or different as the first concentration.

EXAMPLE 37: The method of any examples herein, particularly examples 33–36, wherein the concentration of the pannus limiting material in the each of the n number of sequences is the same or different.

EXAMPLE 38: The method of any examples herein, particularly examples 33–37, wherein the first pannus limiting material, the second pannus limiting material, and the pannus limiting material in the each of the n number of sequences are the same or different and are selected from the pannus limiting material comprises thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

EXAMPLE 39: The method of any examples herein, particularly example 38, wherein the modified thermoplastic polyurethane comprises thermoplastic polyurethane having fluorine and/or silicone incorporated in a polymer structural backbone.

EXAMPLE 40: The method of any examples herein, particularly example 38, wherein the modified thermoplastic polyurethane comprises a mixture of thermoplastic polyurethane and a compound having fluorine-containing moieties and/or silicone.

EXAMPLE 41: The method of any examples herein, particularly example 38, wherein the modified inorganic backbone polymer is modified with halogen-containing groups, phosphorus-containing groups, nitrogen-containing groups, or a combination thereof.

EXAMPLE 42: The method of any examples herein, particularly example 38, wherein the polyethylenes comprise ultra-high-molecular-weight-polyethylene (UHMWPE), low-density polyethylene (LDPE), high density polyethylene (HDPE), or any combination thereof.

EXAMPLE 43: The method of any examples herein, particularly examples 22–42, wherein the one or more sutures are smoothed to substantially remove a surface roughness before attaching the at least a portion of the inner skirt and/or the at least a portion of the leaflet structure, and/or the at least a portion of the outer skirt to at least the annular frame.

EXAMPLE 44: The method of any examples herein, particularly example 43, wherein a step of smoothing comprises heat treating the one or more sutures at a temperature of about 120 °C to about 210 °C and a pressure of about 0.5 MPa to about 2.5 MPa.

EXAMPLE 45: The method of any examples herein, particularly example 22, wherein the method comprises immersing the annular frame with the attached inner skirt into a mixture comprising a pannus limiting material present in a predetermined concentration for a predetermined time to form a second coating.

EXAMPLE 46: The method of any examples herein, particularly example 45, wherein the method comprises an m number of sequences, wherein each sequence comprises the step of immersing, for a further predetermined time, into a further mixture having a further pannus limiting material in a further predetermined concentration, wherein m is from 1 to 10, and wherein at least one applies: the further pannus limiting materials in the each of the m sequences are the same or different; the further predetermined concentrations in the each of the m sequences are the same or different; and/or; the further predetermined times in the each of the m sequences are the same or different.

EXAMPLE 47: The method of any examples herein, particularly example 46, wherein the furtherpannus limiting materials in the each of the m sequences are selected from thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

EXAMPLE 48: The method of any examples herein, particularly example 47, wherein the modified thermoplastic polyurethane comprises thermoplastic polyurethane having fluorine and/or silicone incorporated in a polymer structural backbone.

EXAMPLE 49: The method of any examples herein, particularly example 47, wherein the modified thermoplastic polyurethane comprises a mixture of thermoplastic polyurethane and a compound having fluorine-containing moieties and/or silicone.

EXAMPLE 50: The method of any examples herein, particularly example 47, wherein the modified inorganic backbone polymer is modified with halogen-containing groups, phosphorus-containing groups, nitrogen-containing groups, or a combination thereof.

EXAMPLE 51: The method of any examples herein, particularly example 47, wherein the polyethylenes comprise ultra-high-molecular-weight-polyethylene (UHMWPE), low-density polyethylene (LDPE), high density polyethylene (HDPE), or any combination thereof.

EXAMPLE 52: The method of any examples herein, particularly examples 45-51, wherein if at least a portion of a leaflet structure and/or at least a portion of an outer skirt are attached to at least the annular frame, the at least a portion of the leaflet structure and/or the at least a portion of the outer skirt are masked prior to the step of immersing.

EXAMPLE 53: The method of any examples herein, particularly examples 45–51, further comprises attaching at least a portion of a leaflet structure and/or at least a portion of an outer skirt to at least the annular frame with the one or more sutures.

EXAMPLE 54: The method of any examples herein, particularly examples 45–53, wherein the one or more sutures are treated according to the methods of any one of claims 22–44 prior to the step of attaching.

EXAMPLE 55: The method of any examples herein, particularly examples 24–54, wherein the at least a portion of the inner skirt, and/or the at least a portion of the leaflet structure, and/or the at least a portion of the outer skirt, and the one or more sutures are substantially free of roughness higher than about 0.5 μm.

EXAMPLE 56: The method of any examples herein, particularly examples 24–55, wherein the at least a portion of the inner skirt, and/or the at least a portion of the leaflet structure, and/or the at least a portion of the outer skirt, and the one or more sutures are substantially free of turbulence-inducing surface topography causing a pannus formation.

EXAMPLE 57: The method of any examples herein, particularly examples 24–56, wherein the at least a portion of the inner skirt, and/or the at least a portion of the leaflet structure, and/or the at least a portion of the outer skirt, and the one or more sutures together form a substantially uniform surface profile.

EXAMPLE 58: The method of any examples herein, particularly examples 22–57, wherein the first and/or second coating have a thickness of about 50 nm to about 150 microns.

EXAMPLE 59: A method preventing tissue ingrowth comprising inserting the implantable prosthetic valve of any examples herein, particularly examples 1–21, into a patient’s body.

EXAMPLE 60: A kit comprising one or more sutures configured to attach at least a portion of an inner skirt, and/or at least a portion of a leaflet structure, and/or at least a portion of an outer skirt to at least a frame of an implantable prosthetic valve, wherein the one or more sutures are configured to substantially reduce pannus formation and/or uncontrolled tissue ingrowth along the implantable prosthetic valve.

EXAMPLE 61: The kit of any examples herein, particularly example 60, wherein the kit comprises the one or more sutures having a substantially cylindrical form, a substantially flattened form, or a combination thereof.

EXAMPLE 62: The kit of any examples herein, particularly example 60 or 61, wherein at least a portion of a surface of the one or more sutures has a coating comprising a pannus limiting material.

EXAMPLE 63: The kit of any examples herein, particularly examples 60–62, wherein a substantially entire surface of the one or more sutures has the coating comprising the pannus limiting material.

EXAMPLE 64: The kit of any examples herein, particularly examples 60–63, wherein the pannus limiting material comprises thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

EXAMPLE 65: The kit of any examples herein, particularly example 64, wherein the modified thermoplastic polyurethane comprises thermoplastic polyurethane having fluorine and/or silicone incorporated in a polymer structural backbone.

EXAMPLE 66: The kit of any examples herein, particularly example 64, wherein the modified thermoplastic polyurethane comprises a mixture of thermoplastic polyurethane and a compound having fluorine-containing moieties and/or silicone.

EXAMPLE 67: The kit of any examples herein, particularly example 64, wherein the modified inorganic backbone polymer is modified with halogen-containing groups, phosphorus-containing groups, nitrogen-containing groups, or a combination thereof.

EXAMPLE 68: The kit of any examples herein, particularly example 64, wherein the polyethylenes comprise ultra-high-molecular-weight-polyethylene (UHMWPE), low-density polyethylene (LDPE), high density polyethylene (HDPE), or any combination thereof.

EXAMPLE 69: The kit of any examples herein, particularly examples 60–68, wherein the one or more sutures comprise a polyester, polyethylene, ultrahigh molecular weight polyethylene, silicone, polyurethane, polytetrafluoroethylene, polyether ether ketone (PEEK), fluoropolymer, or natural tissue, or a combination thereof.

EXAMPLE 70: An implantable prosthetic valve comprising: an annular frame having an inner surface and an outer surface wherein the frame has an inflow end and an outflow end, and a central longitudinal axis extending from the inflow end to the outflow end; an inner skirt having an inner surface and an outer surface and positioned along the inner surface of the frame; wherein the inner surface of the inner skirt comprises a first pannus limiting material; an optional leaflet structure having an inner surface and an outer surface and attached toa the inner surface of the annular frame; and/or an optional outer skirt having an inner surface and an outer surface and positioned at the outer surface of the annular frame, and one or more sutures, wherein the one or more sutures are configured to attach at least a portion of the inner skirt, at least a portion of the leaflet structure, and/or at least a portion of the outer skirt, if present, to at least the annular frame and wherein the one or more sutures are configured to substantially reduce pannus formation and/or uncontrolled tissue ingrowth along the implantable prosthetic valve.

EXAMPLE 71: The implantable prosthetic valve of any examples herein, particularly example 70, wherein the one or more sutures have a substantially cylindrical form.

EXAMPLE 72: The implantable prosthetic valve of any examples herein, particularly example 70, wherein the one or more sutures have a substantially flattened form.

EXAMPLE 73: The implantable prosthetic valve of any examples herein, particularly examples 70–72, wherein at least a portion of a surface of the one or more sutures has a coating comprising a pannus limiting material.

EXAMPLE 74: The implantable prosthetic valve of any examples herein, particularly example 73, wherein a substantially entire surface of the one or more sutures has the coating comprising the pannus limiting material.

EXAMPLE 75: The implantable prosthetic valve of any examples herein, particularly examples 70–74, wherein at least a portion of a surface of the one or more sutures has a coating comprising a second pannus limiting material.

EXAMPLE 76: The implantable prosthetic valve of any examples herein, particularly example 75, wherein the first pannus limiting material and the second pannus limiting material are the same or different and is selected from thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

EXAMPLE 77: The implantable prosthetic valve of any examples herein, particularly example 76, wherein the modified thermoplastic polyurethane comprises thermoplastic polyurethane having fluorine and/or silicone incorporated in a polymer structural backbone.

EXAMPLE 78: The implantable prosthetic valve of any examples herein, particularly example 76, wherein the modified thermoplastic polyurethane comprises a mixture of thermoplastic polyurethane and a compound having fluorine-containing moieties and/or silicone.

EXAMPLE 79: The implantable prosthetic valve of any examples herein, particularly example 76, wherein the modified inorganic backbone polymer is modified with halogen-containing groups, phosphorus-containing groups, nitrogen-containing groups, or a combination thereof.

EXAMPLE 80: The implantable prosthetic valve of any examples herein, particularly example 76, wherein the polyethylenes comprise ultra-high-molecular-weight-polyethylene (UHMWPE), low-density polyethylene (LDPE), high density polyethylene (HDPE), or any combination thereof.

In view of the many possible aspects to which the principles of the disclosed disclosure can be applied, it should be recognized that the illustrated aspects are only some examples of the disclosure and should not be taken as limiting the scope of the disclosure. Rather, the scope of the disclosure is defined by the following claims. We, therefore, claim as our disclosure all that comes within the scope and spirit of these claims.

We claim:

1. An implantable prosthetic valve comprising:
an annular frame having an inner surface and an outer surface wherein the frame has an inflow end and an outflow end, and a central longitudinal axis extending from the inflow end to the outflow end;
an inner skirt having an inner surface and an outer surface and positioned along the inner surface of the frame;
an optional leaflet structure having an inner surface and an outer surface and attached to the inner surface of the annular frame; and/or
an optional outer skirt having an inner surface and an outer surface and positioned at the outer surface of the annular frame, and
one or more sutures, wherein the one or more sutures are configured to attach at least a portion of the inner skirt, at least a portion of the leaflet structure, and/or at least a portion of the outer skirt, if present, to at least the annular frame, and wherein the one or more sutures are configured to substantially reduce pannus formation and/or uncontrolled tissue ingrowth along the implantable prosthetic valve.

2. The implantable prosthetic valve of claim 1, wherein:
the one or more sutures have a substantially cylindrical form or
the one or more sutures have a substantially flattened form.

3. The implantable prosthetic valve of claim 1, wherein at least a portion of a surface of the one or more sutures has a coating comprising a pannus limiting material.

4. The implantable prosthetic valve of claim 3, wherein the pannus limiting material comprises thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

5. The implantable prosthetic valve of claim 4, wherein the modified thermoplastic polyurethane comprises:
a thermoplastic polyurethane having fluorine and/or silicone incorporated in a polymer structural backbone or
a mixture of thermoplastic polyurethane and a compound having fluorine-containing moieties and/or silicone.

6. The implantable prosthetic valve of claim 4, wherein the modified inorganic backbone polymer is modified with halogen-containing groups, phosphorus-containing groups, nitrogen-containing groups, or a combination thereof.

7. The implantable prosthetic valve of claim 3, wherein the coating is applied to the one or more sutures:
prior to the attachment of the at least a portion of the inner skirt, and/or the at least a portion of the leaflet structure, and/or the at least a portion of the outer skirt, if present, to at least the annular frame or
after the attachment of the at least a portion of the inner skirt, and/or the at least a portion of the leaflet structure, and/or the at least a portion of the outer skirt, if present, to at least the annular frame.

8. The implantable prosthetic valve of claim 7, wherein the at least a portion of the inner skirt, and/or the at least a portion of the leaflet structure, and/or the at least a portion of the outer skirt, if present, comprises the coating comprising the pannus limiting material.

9. The implantable prosthetic valve of claim 1, wherein the at least a portion of the inner skirt, and/or the at least a portion of the leaflet structure, and/or the at least a portion of the outer skirt, if present, and the one or more sutures are substantially free of roughness higher than about 0.5 μm; and/or
wherein the at least a portion of the inner skirt, and/or the at least a portion of the leaflet structure, and/or the at least a portion of the outer skirt, if present, and the one or more sutures are substantially free of a turbulence-inducing surface topography causing a pannus formation; and/or
wherein the at least a portion of the inner skirt, and/or the at least a portion of the leaflet structure, and/or the at least a portion of the outer skirt, if present, and the one or more sutures together form a substantially uniform surface profile.

10. The implantable valve of claim 3, wherein the coating has a thickness of about 50 nm to about 150 μm.

11. The implantable valve of claim 1, wherein the one or more sutures comprise polyester, polyethylene, ultrahigh molecular weight polyethylene, silicone, polyurethane, polytetrafluoroethylene, polyether ether ketone (PEEK), fluoropolymer, or natural tissue, or a combination thereof.

12. A method of forming an implantable prosthetic valve comprising:
attaching at least a portion of an inner skirt to at least an annular frame with one or more sutures such that an outer surface of the inner skirt is at least partially attached to an inner surface of the annular frame, wherein the one or more sutures are configured to substantially reduce pannus formation along the implantable prosthetic valve, and wherein an inner surface of the inner skirt faces an inner space formed by the annular frame; and
wherein the annular frame has an inflow end and an outflow end, and a central longitudinal axis extending from the inflow end to the outflow end.

13. The method of claim 12, comprising attaching at least a portion of a leaflet structure having an inner surface and an outer surface with the one or more sutures to at least the annular frame.

14. The method of claim 12 , comprising attaching at least a portion of an outer skirt with the one or more sutures to at least the annular frame.

15. The method of claim 12, wherein the one or more sutures are flattened prior to attaching the at least a portion of the inner skirt to at least the annular frame and/or wherein the one or more sutures are flattened prior to attaching the at least a portion of the leaflet structure to at least the annular frame and/or wherein the one or more sutures are flattened prior to attaching the at least a portion of the outer skirt to at least the annular frame, and wherein flattening of the one or more sutures comprises passing the one or more sutures between at least two heat presses at a temperature of about 120 °C to about 210 °C and a pressure of about 0.5 MPa to about 5 MPa.

16. The method of claim 12, wherein the one or more sutures have a first coating comprising a pannus limiting material, and wherein forming the first coating comprises inserting the one or more sutures into a first solution for a first predetermined time, wherein the first solution comprises a first concentration of a first pannus limiting material.

17. The method of claim 16, wherein the step forming the first coating further comprises inserting the one or more sutures into a second solution for a second predetermined time, wherein the second solution comprises a second concentration of a second pannus limiting material.

18. The method of claim 17, wherein the method further comprises an additional n number of sequences, wherein each sequence comprises the step of inserting the one or more sutures, for a predetermined time, into a solution comprising a concentration of a pannus limiting material, and wherein n is from 1 to 10.

19. The method of claim 18, wherein the first pannus limiting material, the second pannus limiting material, and the pannus limiting material in the each of the n number of sequences are the same or different and are selected from the pannus limiting material comprises thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

20. The method of claim 12, wherein the one or more sutures are smoothed to substantially remove a surface roughness before attaching the at least a portion of the inner skirt, and/or the at least a portion of the leaflet structure, and/or the at least a portion of the outer skirt to at least the annular frame, and wherein a step of smoothing comprises heat treating the one or more sutures at a temperature of about 120 °C to about 210 °C and a pressure of about 0.5 MPa to about 2.5 MPa.

21. The method of claim 12, wherein the method comprises immersing the annular frame with the attached inner skirt into a mixture comprising a pannus limiting material present in a predetermined concentration for a predetermined time to form a second coating, and wherein the method comprises an m number of sequences, wherein each sequence comprises the step of immersing, for a further predetermined time, into a further mixture having a further pannus limiting material in a further predetermined concentration, wherein m is from 1 to 10, and wherein at least one applies:
the further pannus limiting materials in the each of the m sequences are the same or different;
the further predetermined concentrations in the each of the m sequences are the same or different; and/or the further predetermined times in the each of the m sequences are the same or different; and wherein the further pannus limiting materials in the each of the m sequences are selected from thermoplastic polyurethane, modified thermoplastic polyurethane, polycarbonate urethane, polyether urethane, polyester urethane, fluoropolymers, fluoro-oligomers, polyethylenes, modified inorganic backbone polymers, polyphospazenes, polyacrylates, polysiloxanes, polyesters, any copolymers thereof, or any combination thereof.

22. The method of claim 21, wherein if at least a portion of a leaflet structure and/or at least a portion of an outer skirt are attached to at least the annular frame, the at least a portion of the leaflet structure and/or the at least a portion of the outer skirt are masked prior to the step of immersing.

23. A method preventing tissue ingrowth comprising inserting the implantable prosthetic valve of claim 1 into a patient’s body.

24. A kit comprising one or more sutures configured to attach at least a portion of an inner skirt and/or at least a portion of a leaflet structure and/or at least a portion of an outer skirt to at least a frame of an implantable prosthetic valve, wherein the one or more sutures are configured to substantially reduce pannus formation and/or uncontrolled tissue ingrowth along the implantable prosthetic valve by having at least a portion of a surface of the one or more sutures to have a coating comprising a pannus limiting material.

25. An implantable prosthetic valve comprising:
an annular frame having an inner surface and an outer surface wherein the frame has an inflow end and an outflow end, and a central longitudinal axis extending from the inflow end to the outflow end;
an inner skirt having an inner surface and an outer surface and positioned along the inner surface of the frame; wherein the inner surface of the inner skirt comprises a first pannus limiting material;
an optional leaflet structure having an inner surface and an outer surface and attached to the inner surface of the annular frame; and/or
an optional outer skirt having an inner surface and an outer surface and positioned at the outer surface of the annular frame, and
one or more sutures, wherein the one or more sutures are configured to attach at least a portion of the inner skirt, at least a portion of the leaflet structure and/or at least a portion of the outer skirt, if present, to at least the annular frame and wherein the one or more sutures are configured to substantially reduce pannus formation and/or uncontrolled tissue ingrowth along the implantable prosthetic valve.
